(19) United States
(12) Patent Application Publication
(10) Pub. No.: US 2026/0000774 A1
(43) Pub. Date: Jan. 1, 2026

(54) Insulin-Like Growth Factor Mimetic Peptide Modified Materials to Modify Cellular Inflammation

(71) Applicant: Washington University, St. Louis, MO (US)

(72) Inventors: Nathaniel Huebsch, St. Louis, MO (US); Xiaohong Tan, St. Louis, MO (US); Lori Setton, St. Louis, MO (US)

(73) Assignee: Washington University, St. Louis, MO (US)

(21) Appl. No.: 19/253,508

(22) Filed: Jun. 27, 2025

Related U.S. Application Data
(60) Provisional application No. 63/664,967, filed on Jun. 27, 2024.

(51) Int. Cl. A61K 47/64; A61K 9/06; A61K 9/50; A61K 35/28; A61K 47/61; A61P 37/04

(52) U.S. Cl. CPC A61K 47/642; A61K 9/06; A61K 9/5036; A61K 9/5052; A61K 35/28; A61K 47/61; A61K 47/645; A61P 37/04

(57) ABSTRACT

Among the various aspects of the present disclosure is the provision of a hydrogel encompassing cell-adhesive and insulin-like growth factor (IGF)-1 mimetic peptides to enhance mesenchymal stem cells (MSC) survival, the secretion of pro-reparative factors, and the ability of these cells to attenuate inflammatory responses.

### CROSS-REFERENCE TO RELATED APPLICATIONS

This application claims the benefit of priority to U.S. Provisional Application Ser. No. 63/664,967 filed on Jun. 27, 2024, which is incorporated herein by reference in its entirety.

### STATEMENT REGARDING FEDERALLY SPONSORED RESEARCH OR DEVELOPMENT

This invention was made with government support under AR077678, AR069588, and HL159094, awarded by the National Institutes of Health (NIH). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020471-US-NP_sequence-listing” created on 23 Jun. 2025; 4,448 bytes). The subject matter of the Sequence Listing is incorporated herein by reference in its entirety.

### FIELD OF THE INVENTION

The present disclosure generally relates to methods of cultivating and administering mesenchymal stem cells using functionalized hydrogel compositions.

### BACKGROUND OF THE INVENTION

Transplanted mesenchymal stem cells (MSCs) are attractive for regenerative medicine for their ability to induce repair and modulate inflammation in host tissue. Preclinical and in vitro studies have shown that soluble factors secreted by MSCs enhance host cell proliferation and migration while combating apoptosis and inflammation. In vivo, transplanted MSC actively and continuously secrete cytokines, growth factors, and extracellular vesicles that circumvent a need to deliver very high doses of individual growth factors and/or vesicles. This ability of transplanted MSC to secrete multiple factors allows these cells to modulate multiple host cell behaviors, ultimately leading to therapeutic benefits. For example, transplanted MSC can inhibit T-cell proliferation by secretion of transforming growth factor beta (TGF-β) and prostaglandin E2 (PGE2), while also supporting tissue repair and regeneration by promoting anti-inflammatory M2 macrophage polarization.

A challenge in MSC therapy is that the intrinsic function of these cells alone is often insufficient for targeted disease treatment. Growth factors can significantly enhance the survival of transplanted MSCs and bolster their therapeutic functions (e.g. cytokine production and extracellular matrix biosynthesis). Prior work has shown that priming MSCs with growth factors prior to transplantation helps dampen host immune cell activation by triggering MSC secretion of trophic factors such as insulin-like growth factor (IGF-1), platelet-derived growth factor (PDGF), TGF-β, and interleukin-6 (IL-6). IGF-1, in particular, has been shown to be well-suited in this regard. For example, virally induced overexpression of IGF-1 in MSCs enhanced their survival under stress conditions (e.g. transplantation into ischemic tissue) by triggering PI3K/Akt signaling. Similarly, pretreating bone marrow-derived MSCs with soluble IGF-1 before transplantation into infarcted rodent heart muscle reduced host-production of pro-inflammatory cytokines tumor necrosis factor (TNF)-α, IL-1, and IL-6 gene expression. Finally, IGF-1 overexpression in human umbilical cord-derived MSC significantly enhanced reno-protective effects in gentamicin-induced acute kidney injury by improving renal function, reducing histological injury and apoptosis, and upregulating genes involved in anti-oxidation, anti-inflammatory responses, and cell migration.

Despite the potential benefits of IGF-1 and other growth factors in enhancing the therapeutic potential of MSC, direct administration of soluble growth factors has led to undesired, dangerous off-target effects. In one widely appreciated example, diffusion of soluble BMP-2 away from the target site has been linked to ectopic bone formation, inflammation, and pain. Likewise, cells transplanted without a biomaterial carrier may also escape the target site and cause unintended side effects and/or injury. For example, MSC transplanted into degenerative intervertebral discs (IVD) without a carrier escaped the transplant site and subsequently triggered osteophyte formation. To mitigate these issues, biomaterial carriers have been developed to entrap cells, and bioactive peptide mimetics of growth factors have in turn been grafted to provide an immobilized source of growth factor signaling to these carriers. For example, peptides derived from BMP-2 have been grafted to alginate gels, and this has conferred an ability of these gels to induce osteogenesis in encapsulated MSC. Similarly, the C domain peptide of IGF-1 has been attached to chitosan gels, which enhanced transplanted MSC viability and improved cells' ability to enhance host tissue repair. Self-assembling peptide gels that incorporate the C-domain of IGF-1 have also been used to enhance MSC survival and augment the ability of transplanted MSC to induce anti-inflammatory macrophage polarization in the context of treating acute kidney injury.

### SUMMARY OF THE INVENTION

Among the various aspects of the present disclosure is the provision of a hydrogel combining cell-adhesive and IGF-1-mimetic peptides to enhance MSC survival, the secretion of pro-reparative factors, and enhance the ability of these cells to attenuate inflammatory responses. The peptides may also enhance survival, and attenuate inflammatory responses, of other cell populations such as macrophages.

The present teachings include a hydrogel comprised of an alginate polymer which can be unmodified or conjugated to a peptide comprising a cyclic RGD (cRGD) peptide, an amino acid sequence comprising LCQSWGVRIGWLAGLCPKK (IGM3), or any combination thereof. An aspect of the present disclosure provides for crosslinking the alginate polymer solution using calcium sulfate. In one aspect, the conjugated peptide may be at a concentration of 10 μM or 50 μM. In another aspect, the hydrogel may encapsulate MSCs by combining a volume of unmodified or conjugated alginate polymer with an equal volume of MSCs and crosslinking the polymers with calcium sulfate.

The present teachings include methods for treating a disease or condition comprising administering, to a subject in need, a therapeutically effective amount of a stem cell therapy comprising a hydrogel, an IGF-1 mimetic, a cell adhesive ligand, and an MSC. In one aspect, the hydrogel may be a functionalized alginate hydrogel. In another aspect, the functionalized alginate hydrogel may have a final concentration of 1% w/v alginate. In another aspect, the IGF-1 mimetic may comprise the amino acids LCQSWGVRIGWLAGLCPKK (IGM3). In another aspect, the cell adhesive ligand may be an integrin-binding peptide. In yet another aspect, the integrin-binding peptide may be a cRGD peptide. In another aspect, the disease or condition may be intervertebral disc degeneration.

Another embodiment of the invention includes a method of enhancing stem cell function comprising encapsulating MSCs in an alginate hydrogel conjugated to a peptide. In one aspect, the alginate hydrogel conjugated peptide may be a cRGD peptide, an amino acid sequence comprising LCQSWGVRIGWLAGLCPKK (IGM3), or any combination thereof. In another aspect, the conjugated alginate hydrogel encapsulating MSCs reduces the production of a pro-inflammatory cytokine. In another aspect, the pro-inflammatory cytokines may be TNF-α or GM-CSF. In another aspect, the conjugated alginate hydrogel encapsulating MSCs may increase stem cell survival. In another aspect, the conjugated alginate hydrogel encapsulating MSCs may be used to treat intervertebral disc degeneration. In another aspect, the concentration of the cRGD peptide in the hydrogel may range from 10μM to 10 μM. In another aspect, the concentration of the IGF-1 peptide mimetic in the hydrogel may range from 10 μM to 10 μM.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic diagram showing the structure of Insulin-like growth factor peptide mimetics (IGM-1, IGM-2, IGM-3) as candidate molecules. IGM-1 represents the c-domain of the full-length IGF-1. In contrast, IGM-2 and IGM-3 are peptide mimetics that were identified through phage-display studies.

FIG. 1B is a schematic diagram depicting the conjugation techniques and hydrogel formation. Maleimide-thiol Michael-type addition reaction for IGM peptide conjugation and strain-promoted alkyne-azide cycloaddition for cRGD peptide conjugation are illustrated. Maleimide-IGM-alginate was combined with BCN-Az-cRGD-alginate at various peptide concentrations, and then ionically crosslinked with divalent calcium ions to form a combination peptide (cRGD/IGM) alginate hydrogel.

FIG. 2A is a graph showing resazurin reduction quantification in MSCs cultured in serum-free medium across different hydrogel formulations (alginate, alginate+rhIGF-1 [500 ng/mL], cRGD-alginate, cRGD-alginate+rhIGF-1, and alginate modified with IGM peptides [IGM-1, IGM-2, IGM-3], each combined with cRGD on Day 7, normalized to Day 1 values. Data points represent biological replicates with error bars indicating ±SD.

FIG. 2B is a graph comparing resazurin reduction in MSCs encapsulated in unmodified alginate, cRGD-alginate, or varied peptide density of cRGD and IGM-3 on Day 7, normalized to Day 1 values. Data points represent biological replicates with error bars indicating ±SD.

FIG. 2C is a graph of Akt phosphorylation levels (pAkt/Total Akt) measured two hours post-cell attachment on cRGD-alginate supplemented with rhIGF-1 (500 ng/mL), indicating activation similar to cRGD/IGM-3 alginate. Data points represent biological replicates with error bars indicating ±SD.

FIG. 2D is a graph showing the effect of IGF-I receptor inhibitor (AZ7550 Mesylate, 20 μM) on Akt activity in hydrogels containing rhIGF-1 or IGM-3, demonstrating receptor-specific binding of IGM-3.

FIG. 2E is a graph showing the effect of IGF-1 & insulin receptor inhibitor (GSK1904529A, 3 μM) on Akt activity in hydrogels containing rhIGF-1 or IGM-3, demonstrating receptor-specific binding of IGM-3. Data points represent biological replicates with error bars indicating ±SD.

FIG. 2F is a graph showing the impact of PI3K/Akt pathway inhibition (LY294002, 10 μM) on Akt phosphorylation in hydrogels supplemented with rhIGF-1 and cRGD/IGM-3 alginate after 2 hours. Data points represent biological replicates with error bars indicating ±SD.

FIG. 2G is a graph showing ERK activity modulation by the ERK 1/2 pathway inhibitor (U0126, 10 μM) in MSCs cultured on cRGD alginate, showing pathway involvement in cRGD-mediated effects. Data points represent biological replicates with error bars indicating ±SD.

FIG. 3A is a schematic diagram illustrating the experimental setup used to collect secreted medium from MSC hydrogel cultures for quantification.

FIG. 3B is a graph comparing the total TNFα secretion in MSC supernatants. MSCs were encapsulated in various hydrogel conditions (cRGD/IFG-1) and exposed to 1 ng/mL IL-1β for 4 days. IL-1β priming increased cytokine secretion in all hydrogel conditions, with cRGD and IGM-3 showing a notable reduction in inflammatory cytokines. Data represent biological replicates; error bars denote ±SD.

FIG. 3C is a graph comparing the total GM-CSF secretion in MSC supernatants. MSCs were encapsulated in various hydrogel conditions (cRGD/IGF-1) and exposed to 1 ng/mL IL-1β for 4 days. IL-1β priming increased cytokine secretion in all hydrogel conditions, with cRGD and IGM-3 showing a notable reduction in inflammatory cytokines. Data represent biological replicates; error bars denote ±SD.

FIG. 3D is a graph comparing total TIMP-1 secretion in MSC supernatants. MSCs were encapsulated in various hydrogel conditions (cRGD/IGF-1) and exposed to 1 ng/mL IL-1β for 4 days. IL-1β increased tissue inhibitor of metalloproteinase secretion in MSCs. Data represent biological replicates; error bars denote ±SD.

FIG. 3E is a graph comparing total TIMP-2 secretion in MSC supernatants. MSCs were encapsulated in various hydrogel conditions (cRGD/IGF-1) and exposed to 1 ng/mL IL-1β for 4 days. IL-1β increased tissue inhibitor of metalloproteinase secretion in MSCs. Data represent biological replicates; error bars denote ±SD.

FIG. 3F is a graph comparing total TIMP-4 secretion in MSC supernatants. MSCs were encapsulated in various hydrogel conditions (cRGD/IGF-1) and exposed to 1 ng/mL IL-1β for 4 days. IL-1β increased tissue inhibitor of metalloproteinase secretion in MSCs. Data represent biological replicates; error bars denote ±SD.

FIG. 4A is a schematic diagram illustrating the experimental setup used to collect secreted medium from MSC hydrogel cultures for quantification.

FIG. 4B is a graph of TNFα secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Secreted pro-inflammatory TNF-α levels significantly increased after IL-1β priming and the dual-peptide hydrogel containing cRGD and IGM-3 significantly lowered secreted TNF-α. Data represent biological replicates; error bars denote ±SD.

FIG. 4C is a graph of GM-CSF secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Secreted pro-inflammatory GM-CSF levels significantly increased after IL-1β priming and the dual-peptide hydrogel containing cRGD and IGM-3 significantly lowered secreted GM-CSF. Data represent biological replicates; error bars denote ±SD.

FIG. 4D is a graph of IL-6 secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. IL-6 levels remained consistent across all hydrogel conditions, except for a slight increase in cRGD supplemented with 500 ng/mL rhIGF-1. Data represent biological replicates; error bars denote ±SD.

FIG. 4E is a graph of IL-8 secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Data represent biological replicates; error bars denote ±SD.

FIG. 4F is a graph of IFNγ secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Data represent biological replicates; error bars denote ±SD.

FIG. 4G is a graph of IL-17A secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Data represent biological replicates; error bars denote ±SD.

FIG. 4H is a graph of IL-10 secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. IL-1β priming led to an increase in the secretion of cytokines associated with anti-inflammation. Data represent biological replicates; error bars denote ±SD.

FIG. 4I is a graph of IL-1β secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. IL-1β priming led to an increase in the secretion of cytokines associated with anti-inflammation. Data represent biological replicates; error bars denote ±SD.

FIG. 4J is a graph of TGFβ-1 secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Secreted TGFβ-1 levels were lower in the 10 μM cRGD/50 μM IGM-3 alginate compared to the 50 μM cRGD-only hydrogel. Data represent biological replicates; error bars denote ±SD.

FIG. 4K is a graph of TGFβ-2 secretions from MSCs encapsulated in alginate, cRGD-alginate, cRGD-alginate supplemented with soluble IGF-1 (500 ng/mL, rhIGF-1), or various densities of cRGD/IGM-3 after 1 ng/mL IL-1β challenge. Secreted TGFβ-2 levels were lower in the 10 μM cRGD/50 μM IGM-3 alginate compared to the 50 μM cRGD-only hydrogel. Data represent biological replicates; error bars denote ±SD.

FIG. 5A is a flow chart depicting a bulk RNA-sequencing study. Differential regulation of inflammatory genes in degenerative primary intervertebral disc cells co-cultured with MSCs in cRGD/IGM3 was evaluated.

FIG. 5B is a Principal Component Analysis (PCA) graph showing separate sample groups based on variances: monolayer disc cells (n=4; yellow, triangle), disc cells co-cultured with MSCs in unmodified alginate (MSC-A1g, n=6; green, square), in cRGD alginate (MSC-cRGD, n=6; blue, diamond), and in cRGD/IGM3 alginate (MSC-cRGD/IGM-3, n=6; pink, circle). Overlaps in PCA space indicate similar modulatory effects of MSCs in alginate and cRGD alginate on disc cells.

FIG. 5C is a volcano plot showing differentially regulated genes in disc cells co-cultured with MSCs in alginate compared to monolayer culture, using a cutoff of FDR-adjusted p-value <0.05 and a log 2 fold change >2 or <-2.

FIG. 5D is a volcano plot showing differentially regulated genes in disc cells co-cultured with MSCs in cRGD alginate compared to monolayer culture, using a cutoff of FDR-adjusted p-value <0.05 and a log 2 fold change >2 or <−2.

FIG. 5E is a volcano plot showing differentially regulated genes in disc cells co-cultured with MSCs in cRGD/IGM-3 alginate compared to monolayer culture, using a cutoff of FDR-adjusted p-value <0.05 and a log 2 fold change >2 or <−2.

FIG. 5F is a heatmap of z-score normalized gene expression showing patterns of down-regulated and upregulated genes in disc cells co-cultured with MSC-cRGD/IGM3 versus monolayer

FIG. 5G is a summary of a Gene Ontology (GO) analysis of the most down-regulated molecular function terms, with a cutoff of log 2 fold-change >2 or <−2 and adjusted FDR p-value <0.05.

FIG. 5H is a heatmap showing gene expression profiles mapping to the top three most down-regulated GO terms in the cRGD/IGM-3 condition compared to a monolayer.

FIG. 6 is a graph comparing resazurin reduction in MSCs encapsulated in IGM-3 alginate or varied peptide density of cRGD and IGM-3 on Day 7, normalized to Day 1 values. Data points represent biological replicates with error bars indicating ±SD. Statistical analyses were performed using one-way ANOVA with Tukey's multiple comparisons tests.

FIG. 7A is a schematic diagram illustrating an experimental setup to measure metabolic activity of cells in 3D hydrogels. Primary NP cells or MSCs are encapsulated in peptide-conjugated hydrogel containing serum. At 24 h, media for NP cells and MSCs is replenished with 1% FBS+1% ITS or RoosterBasal, respectively. Metabolic activity (Alamar Blue) was measured at days 1, 3, and 7.

FIG. 7B is a graph comparing the IGF-1 mimetic peptide promotion of metabolic activity in nucleus pulposus (NP) cells. The combination of cRGD/IGM3 peptide promoted NP cell metabolic activity compared to unmodified alginate cultured in media containing 1% FBS and 1% ITS.

FIG. 7C is a graph comparing the IGF-1 mimetic peptide promotion of MSC proliferation. The combination of cRGD/IGM2 or cRGD/IGM3 peptide improved MSC metabolic activity compared to unmodified alginate and cRGD-alginate culture in serum-free media at 7 days.

FIG. 7D is a schematic diagram showing the experimental setup to evaluate the effects of IL-1β on cells encapsulated in 3D hydrogels. Primary NP cells are transduced with lentivirus carrying an NF-κB response element driving the luciferase gene. Firefly luciferase activity is obtained as a measure of NF-κB luciferase levels (Bright-glow™ assay) at 24 h.

FIG. 7E is a graph comparing NF-κB activity of IL-1β treated NP cells in 3D hydrogel (cRGD/IGF-1). The IGF-1 mimetic peptides grafted to alginate hydrogels blunt IL-1β induced inflammatory signaling in NP cells.

FIG. 7F is a graph comparing NF-κB activity of IL-1 treated MSCs in 3D hydrogel (cRGD/IGF-1).

FIG. 8A is a graph comparing TNFα cytokine production in MSCs encapsulated in alginate, cRGD-alginate, cRGD-alginate supplemented with soluble IGF-1 and challenged with IL-1β at day 4.

FIG. 8B is a graph comparing IL-1β cytokine production in MSCs encapsulated in alginate, cRGD-alginate, cRGD-alginate supplemented with soluble IGF-1 and challenged with IL-1β at day 4.

FIG. 8C is a graph comparing CM-CSF cytokine production in MSCs encapsulated in alginate, cRGD-alginate, cRGD-alginate supplemented with soluble IGF-1 and challenged with IL-1β at day 4.

FIG. 8D is a graph comparing IFNγ cytokine production in MSCs encapsulated in alginate, cRGD-alginate, cRGD-alginate supplemented with soluble IGF-1 and challenged with IL-1β at day 4.

FIG. 8E is a graph comparing IL-10 cytokine production in MSCs encapsulated in alginate, cRGD-alginate, cRGD-alginate supplemented with soluble IGF-1 and challenged with IL-1β at day 4.

FIG. 8F is a graph comparing IL-12p70 cytokine production in MSCs encapsulated in alginate, cRGD-alginate, cRGD-alginate supplemented with soluble IGF-1 and challenged with IL-1β at day 4.

FIG. 9A is a graph comparing total AKT in MSCs encapsulated in alginate, cRGD-alginate, cRGD-alginate supplemented with IGM3 peptide, and cRGD-alginate supplemented with soluble IGF-1. The addition of the PI3K/Akt pathway inhibition (LY294002) decreases total AKT levels.

FIG. 9B is a graph comparing pAKT in MSCs encapsulated in alginate, cRGD-alginate, cRGD-alginate supplemented with IGM3 peptide, and cRGD-alginate supplemented with soluble IGF-1. The addition of the PI3K/Akt pathway inhibition (LY294002) decreases pAKT levels.

FIG. 9C is a graph comparing pAKT/AKT in MSCs encapsulated in alginate, cRGD-alginate, cRGD-alginate supplemented with IGM3 peptide, and cRGD-alginate supplemented with soluble IGF-1. The addition of the PI3K/Akt pathway inhibition (LY294002) decreases pAKT/AKT levels.

FIG. 10A is a schematic diagram illustrating intervertebral discs across stages of degeneration (left, healthy; middle, early degeneration; right, late degeneration). NP-nucleus pulposus, AP-articular processes. IVD-intervertebral disc.

FIG. 10B is a graph comparing cellular proliferation in injured mouse spinal cords following BMMSC (left) and VMMSC-IGF1 transplantation.

FIG. 11 is a schematic diagram illustrating ECM and growth factor mimetic peptides grafted via bio-orthogonal click chemistry. Co-presenting cell adhesive ligands are linked with growth factor-mimetic peptides on alginate to deliver growth factors in a controlled manner.

FIG. 12 is a schematic diagram illustrating the localized presentation of tethered cell adhesive ang growth factor mimetic peptides via hydrogel compared to growth factor introduced in solution.

### DETAILED DESCRIPTION OF THE INVENTION

The present disclosure is based, at least in part, on the discovery that IGF-1 peptide mimetics enhance the survival and immunomodulatory capabilities of MSCs.

A bioactive IGF-1 peptide mimetic identified in a screen proved much more potent than the C-domain of IGF-1 in improving MSC survival under serum-deprived culture conditions. Survival was improved markedly beyond what we observed in MSC cultured in gels presented with cell adhesive cyclic RGD (cRGD) peptides alone, suggesting a potentially unique role for growth factor mimetics in promoting cell survival. Interestingly, although this new peptide does not share sequence similarity with IGF-1, it activated Akt signaling in a manner similar to the full-length growth factor and significantly blunted the inflammatory response of MSC provoked with IL-1β. In co-culture studies, MSCs encapsulated within the alginate hydrogel functionalized with both the IGF-1 peptide mimetic and cRGD markedly blunted inflammatory responses in primary cells retrieved from degenerative human IVDs. The combination of cell-adhesive and IGF-1-mimetic peptides not only enhances MSC survival and the secretion of pro-reparative factors but also enhances the ability of these cells to attenuate inflammatory responses.

In one aspect, the hydrogel may be an unmodified alginate hydrogel or a functionalized hydrogel. In one aspect, the alginate hydrogel may be functionalized with cRGD peptides, IGF-1, or an IGF-1 mimetic. In another aspect, the IFG-1 mimetic may be IGM-1 (SEQ ID NO:1), IGM-2 (SEQ ID NO:2), or IGM-3 (SEQ ID NO:3).

The concentration of the cRGD peptide and the IGF-1 mimetic in the hydrogel may independently range from about 10 μM to about 50 μM. In some aspects, the hydrogel may contain an unmodified alginate, 10 μM cRGD-alginate, 50 μM cRGD-alginate, 50 μM cRGD/50 μM IGM-1, 50 μM cRGD/50 μM IGM-2, 50 μM cRGD/50 μM IGM-3, or 10 μM cRGD/50 μM IGM-3.

In some aspects, the hydrogel may be functionalized with a peptide that activates the PI3K/Akt pathway and/or the JAK/STAT pathway.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Therapeutic Methods

Also provided is an example of a process of treating a disease or condition in a subject in need of administration of a therapeutically effective amount of stem cells encapsulated in a functionalized hydrogel, so as to promote cell growth and reduce inflammation.

Methods described herein are generally performed on a subject in need thereof. A subject in need of the therapeutic methods described herein can be a subject having, diagnosed with, suspected of having, or at risk for developing a disease or condition characterized by inflammation or tissue degeneration or damage. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, chickens, and humans. For example, the subject can be a human subject.

Generally, a safe and effective amount of stem cells encapsulated in a functionalized hydrogel, for example, that amount that would cause the desired therapeutic effect in a subject while minimizing undesired side effects. In various embodiments, an effective amount of stem cells encapsulated in a functionalized hydrogel described herein can substantially inhibit inflammation, promote tissue repair, and promote cell proliferation.

According to the methods described herein, administration can be parenteral, inhaled, oral, topical, intradermal, intramuscular, intraperitoneal, intravenous, subcutaneous, intranasal, epidural, ophthalmic, buccal, or rectal administration.

When used in the treatments described herein, a therapeutically effective amount of stem cells encapsulated in a functionalized hydrogel can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to promote cell growth or reduce inflammation.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of stem cells encapsulated in a functionalized hydrogel, or delivery of the hydrogel alone without cells, can occur as a single event or over a time course of treatment. For example, a functionalized hydrogel can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for tissue repair and regeneration.

A functionalized hydrogel can be administered simultaneously or sequentially with another agent, such as an antibiotic, an anti-inflammatory, or another agent. For example, a functionalized hydrogel can be administered simultaneously with another agent, such as an antibiotic or an anti-inflammatory. Simultaneous administration can occur through the administration of separate compositions, each containing one or more of a functionalized hydrogel, an antibiotic, an anti-inflammatory, or another agent. Simultaneous administration can occur through the administration of one composition containing two or more of a functionalized hydrogel, an antibiotic, an anti-inflammatory, or another agent. A functionalized hydrogel can be administered sequentially with an antibiotic, an anti-inflammatory, or another agent. For example, a functionalized hydrogel can be administered before or after the administration of an antibiotic, an anti-inflammatory, or another agent.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: IGF-1 Peptide Mimetic-Functionalized Hydrogels Enhance MSC Survival and Immunomodulatory Activity

Human mesenchymal stem cells (MSCs) have demonstrated promise when delivered to damaged tissue or tissue defects for their cytokine secretion and inflammation modulation behaviors that can promote repair. Insulin-like growth factor 1 (IGF-1) has been shown to augment MSCs' viability and survival and promote their secretion of cytokines that signal to endogenous cells, in the treatment of myocardial infarction, wound healing, and age-related diseases. Biomaterial cell carriers can be functionalized with growth factor-mimetic peptides to enhance MSC function while promoting cell retention and minimizing off-target effects seen with direct administration of soluble growth factors. Here, we functionalized alginate hydrogels with three distinct IGF-1 peptide mimetics and the integrin-binding peptide, cyclic RGD. One IGF-1 peptide mimetic (IGM-3) was found to activate Akt signaling and support the survival of serum-deprived MSCs. MSCs encapsulated in alginate hydrogels that presented both IGM-3 and cRGD showed a significant reduction in pro-inflammatory cytokine secretion when challenged with interleukin-1 b. Finally, MSCs cultured within the cRGD/IGM-3 hydrogels were able to blunt pro-inflammatory gene expression of human primary cells from degenerated intervertebral discs. These studies indicate the ability to leverage cell adhesive and IGF-1 growth factor peptide mimetics together to control the therapeutic secretory behavior of MSCs.

### Introduction

Transplanted mesenchymal stem cells (MSCs) are attractive for regenerative medicine for their ability to induce repair and modulate inflammation in host tissue. Preclinical and in vitro studies have shown that soluble factors secreted by MSCs enhance host cell proliferation and migration, while combating apoptosis and inflammation. In vivo, transplanted MSC actively and continuously secrete cytokines, growth factors and extracellular vesicles that circumvent a need to deliver very high doses of individual growth factors and/or vesicles. This ability of transplanted MSC to secrete multiple factors allows these cells to modulate multiple host cell behaviors, ultimately leading to therapeutic benefits. For example, transplanted MSC can inhibit T-cell proliferation by secretion of transforming growth factor beta (TGF-β) and prostaglandin E2 (PGE2), while also supporting tissue repair and regeneration by promoting anti-inflammatory M2 macrophage polarization.

A challenge in MSC therapy is that the intrinsic function of these cells alone is often insufficient for targeted disease treatment. Growth factors can significantly enhance the survival of transplanted MSCs and also bolster their therapeutic functions (e.g. cytokine production and extracellular matrix biosynthesis). Prior work has shown that priming MSCs with growth factors prior to transplantation helps dampen host immune cell activation by triggering MSC secretion of trophic factors such as insulin-like growth factor (IGF-1), platelet-derived growth factor (PDGF), TGF-β, and interlukin-6 (IL-6). IGF-1 has been shown to be well-suited in this regard. For example, virally-induced overexpression of IGF-1 in MSCs enhanced their survival under stress conditions (e.g. transplantation into ischemic tissue) by triggering PI3K/Akt signaling. Similarly, pretreating bone marrow-derived MSCs with soluble IGF-1 before transplantation into infarcted rodent heart muscle reduced host-production of pro-inflammatory cytokines TNF-α, IL-1, and IL-6 gene expression. Finally, IGF-1 overexpression in human umbilical cord-derived MSC significantly enhanced renoprotective effects in gentamicin-induced acute kidney injury by improving renal function, reducing histological injury and apoptosis, and upregulating genes involved in anti-oxidation, anti-inflammatory responses, and cell migration.

Despite the potential benefits of IGF-1 and other growth factors in enhancing the therapeutic potential of MSC, direct administration of soluble growth factors has led to undesired, dangerous off-target effects. In one widely appreciated example, diffusion of soluble bone morphogenic protein 2 (BMP-2) away from the target site has been linked to ectopic bone formation, inflammation and pain. Likewise, cells transplanted without a biomaterial carrier may also escape the target site and cause intended side effects and/or injury. For example, MSC transplanted into degenerative intervertebral discs (IVD) without a carrier escaped the transplant site and subsequently triggered osteophyte formation. To mitigate these issues, biomaterial carriers have been developed to entrap cells, and bioactive peptide mimetics of growth factors have in turn been grafted to provide an immobilized source of growth factor signaling to these carriers. For example, peptides derived from BMP-2 have been grafted to alginate gels, and this has conferred an ability of these gels to induce osteogenesis in encapsulated MSC. Similarly, the C domain peptide of IGF-1 has been attached to chitosan gels, which enhanced transplanted MSC viability and improved cells' ability to enhance host tissue repair. Self-assembling peptide gels that incorporate the C-domain of IGF-1 have also been used to enhance MSC survival and augment the ability of transplanted MSC to induce anti-inflammatory macrophage polarization in the context of treating acute kidney injury.

Here, we sought to use IGF-1 peptide mimetics to enhance survival and immunomodulatory capabilities of MSC. In a screen of three candidate peptides, a bioactive IGF-1 peptide mimetic that was identified previously through phage display proved much more potent than the C-domain of IGF-1 in improving MSC survival under serum-deprived culture conditions. Survival was improved markedly beyond what we observed in MSC cultured in gels presented with cell adhesive cyclic RGD (cRGD) peptides alone, suggesting a potential unique role for growth factor mimetics in promoting cell survival. Interestingly, although this new peptide does not share sequence similarity with IGF-1, it activated Akt signaling in a manner similar to the full-length growth factor, and significantly blunted the inflammatory response of MSC provoked with IL-1β. In co-culture studies, MSCs encapsulated within the alginate hydrogel functionalized with both the IGF-1 peptide mimetic and cRGD markedly blunted inflammatory responses in primary cells retrieved from degenerative human IVDs. Our findings indicate that the combination of cell-adhesive and IGF-1-mimetic peptides not only enhances MSC survival and the secretion of pro-reparative factors but also enhances the ability of these cells to attenuate inflammatory responses.

### Results

### Effects of Growth Factor-Mimetic Peptide Grafted Alginate on Encapsulated MSCs

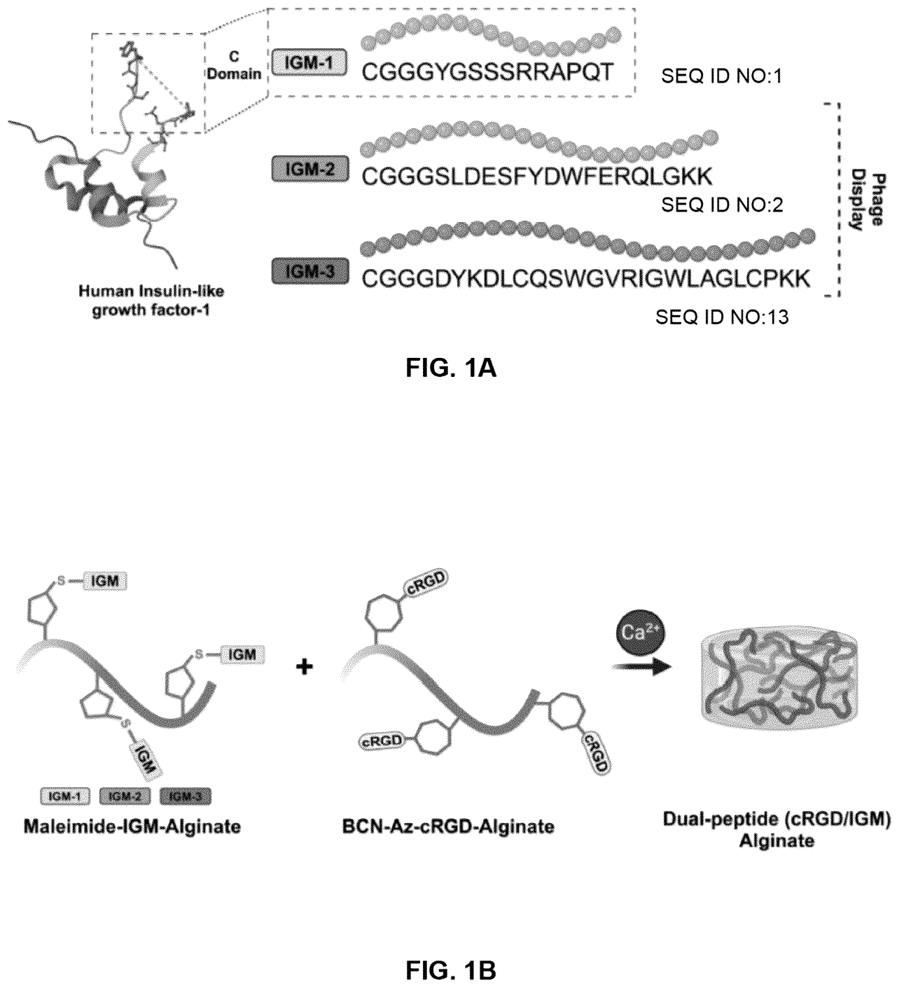

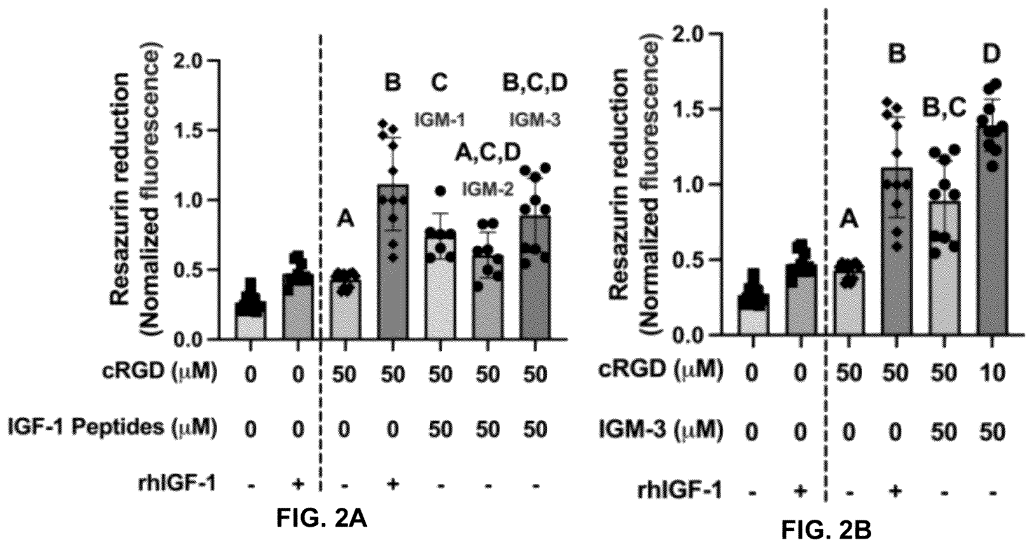

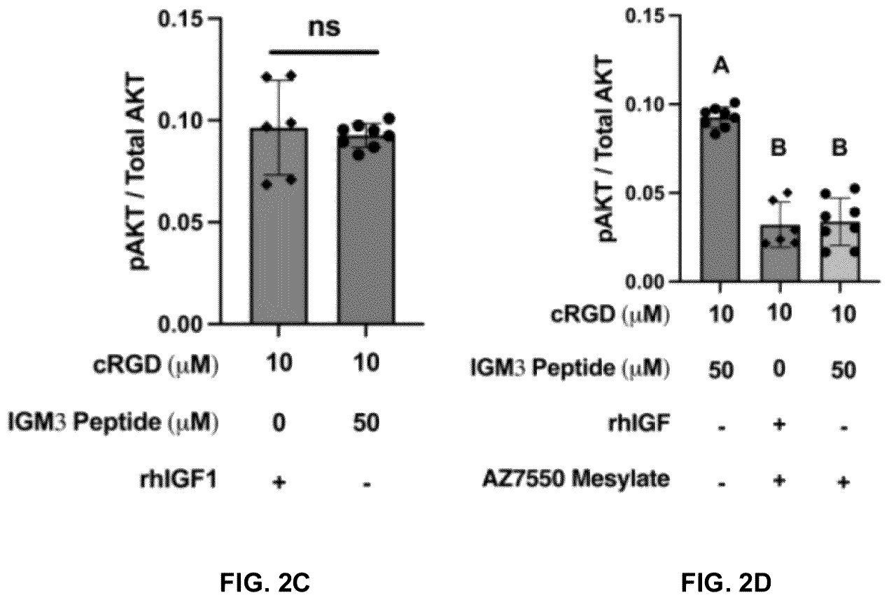

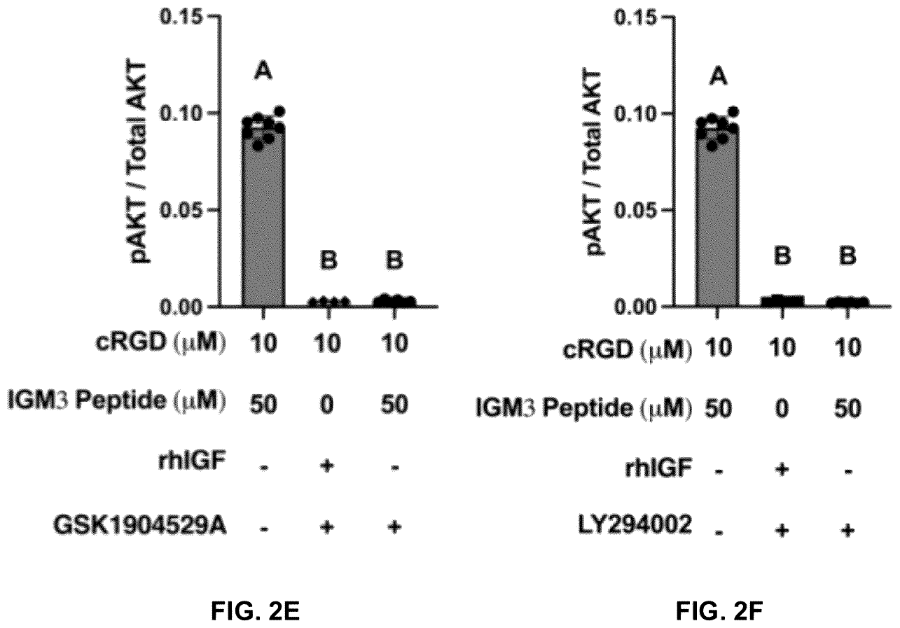

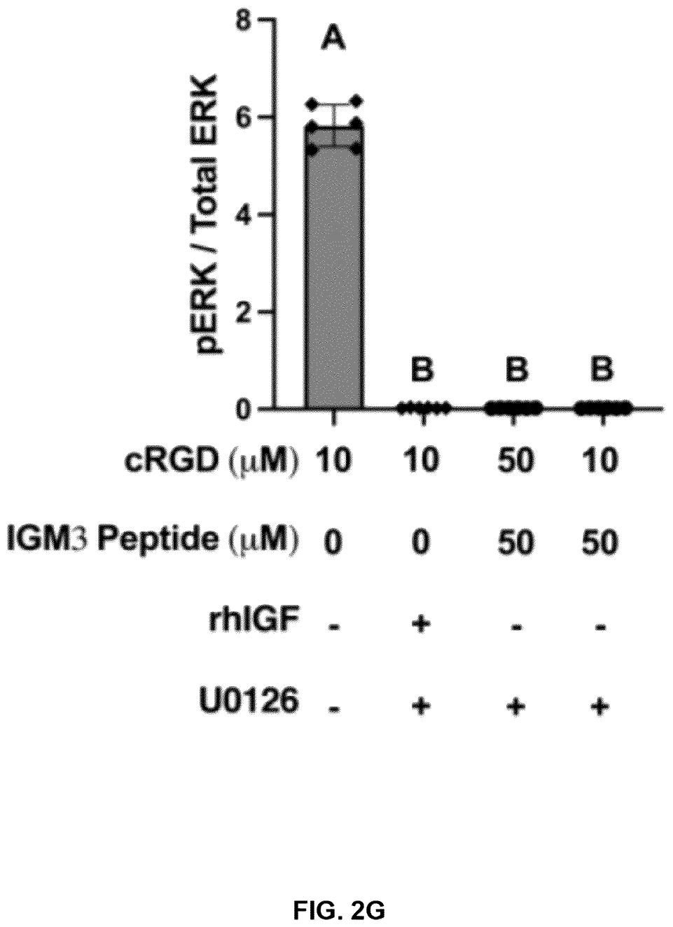

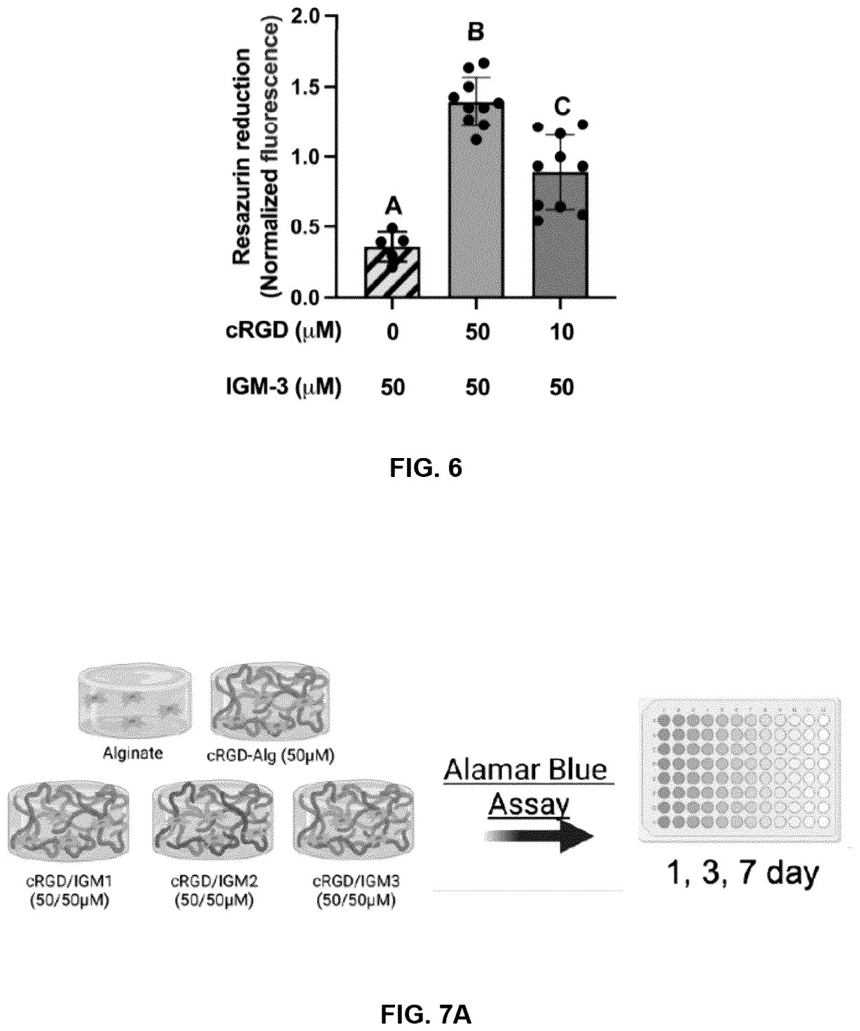

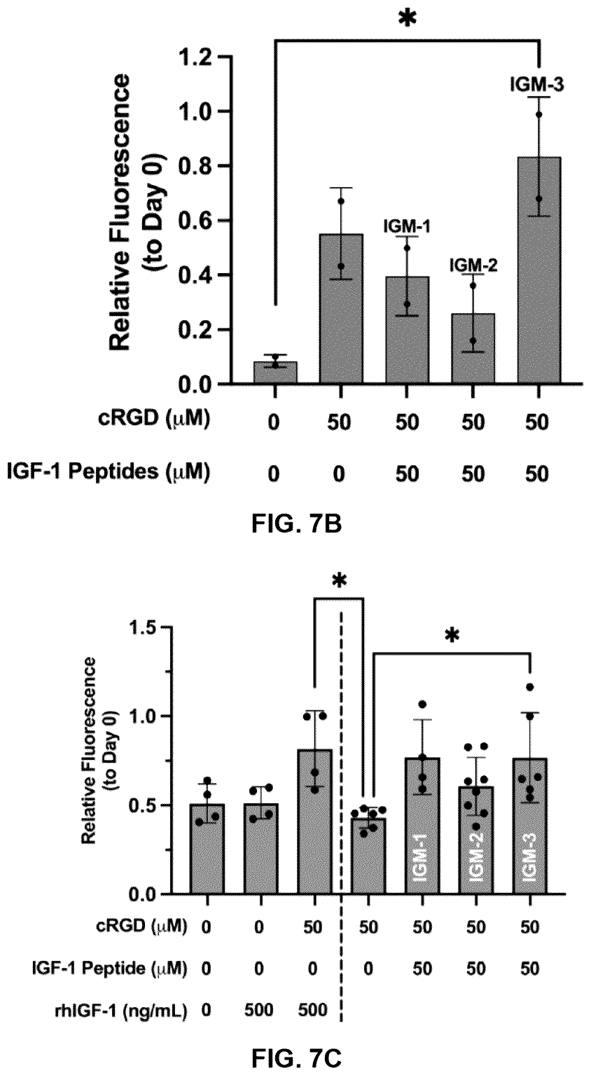

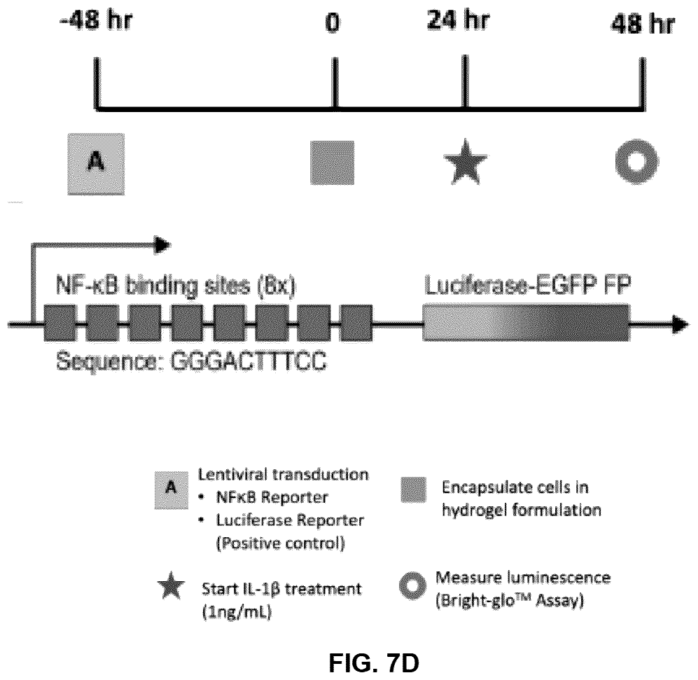

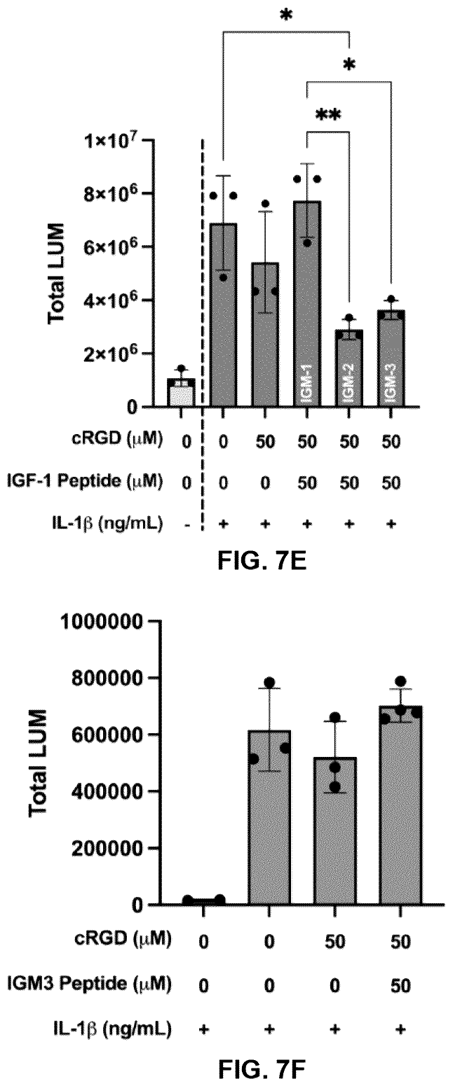

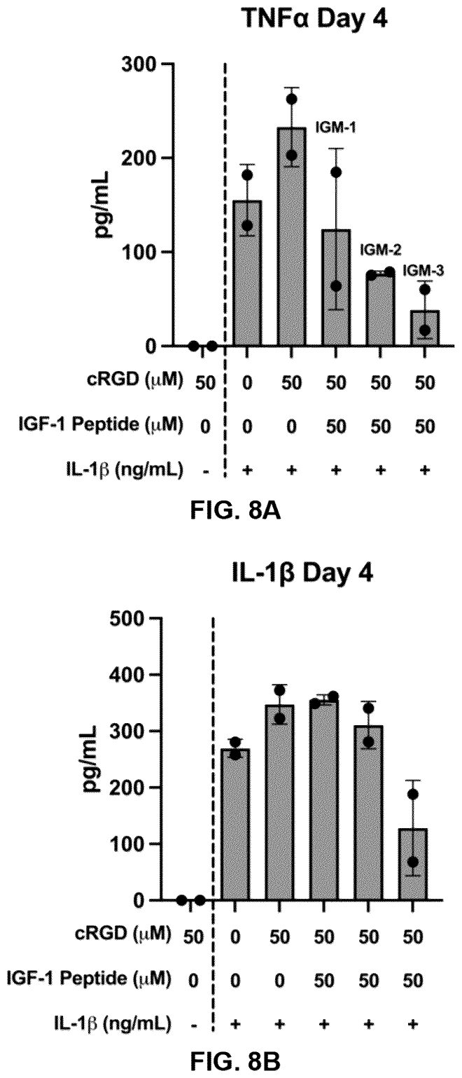

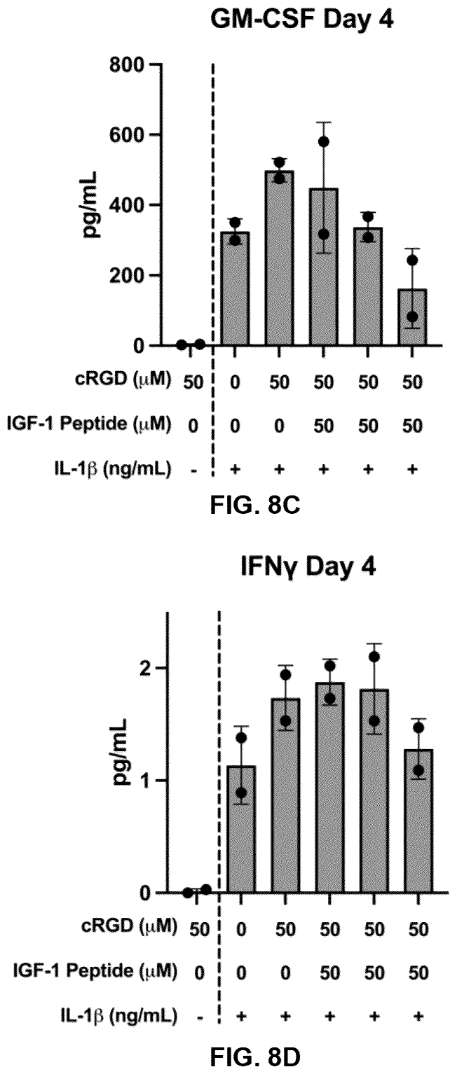

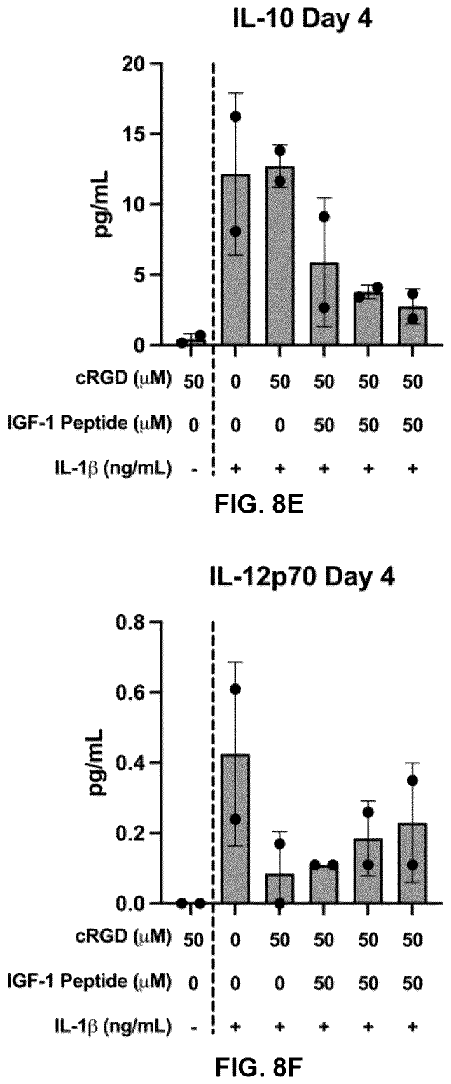

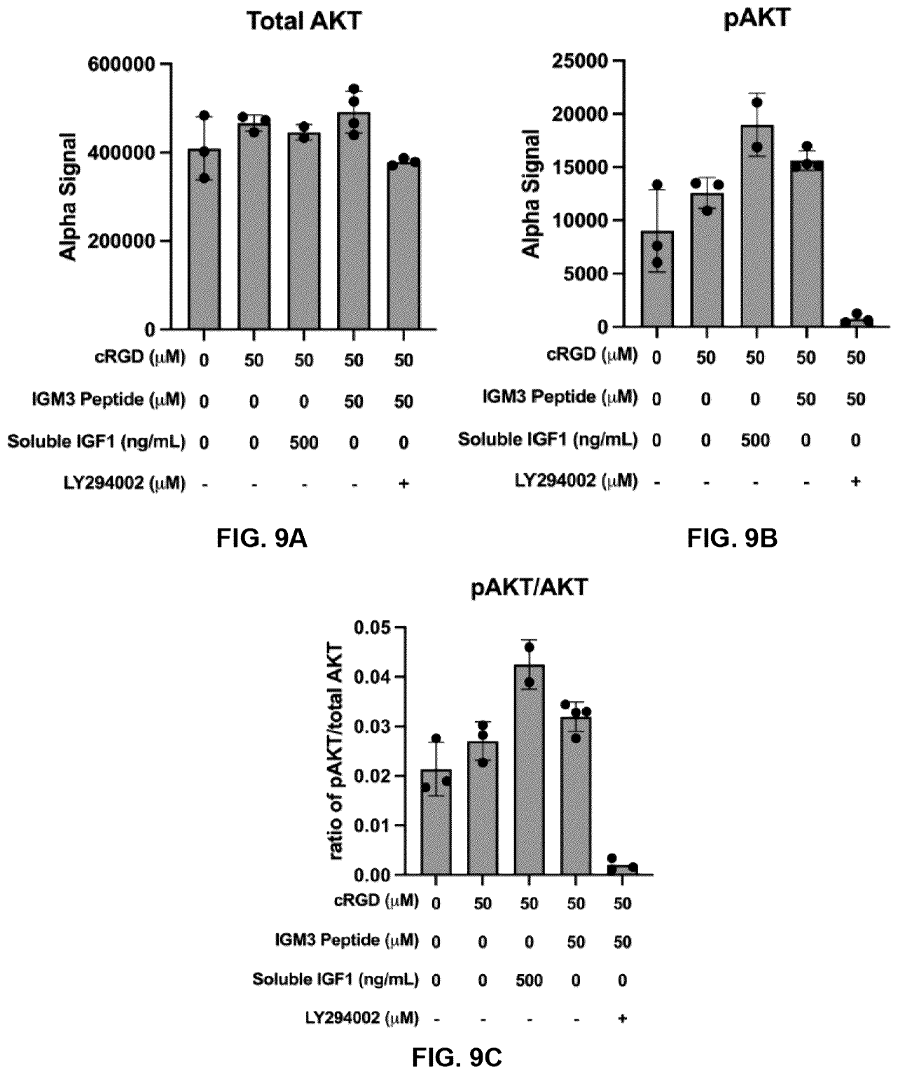

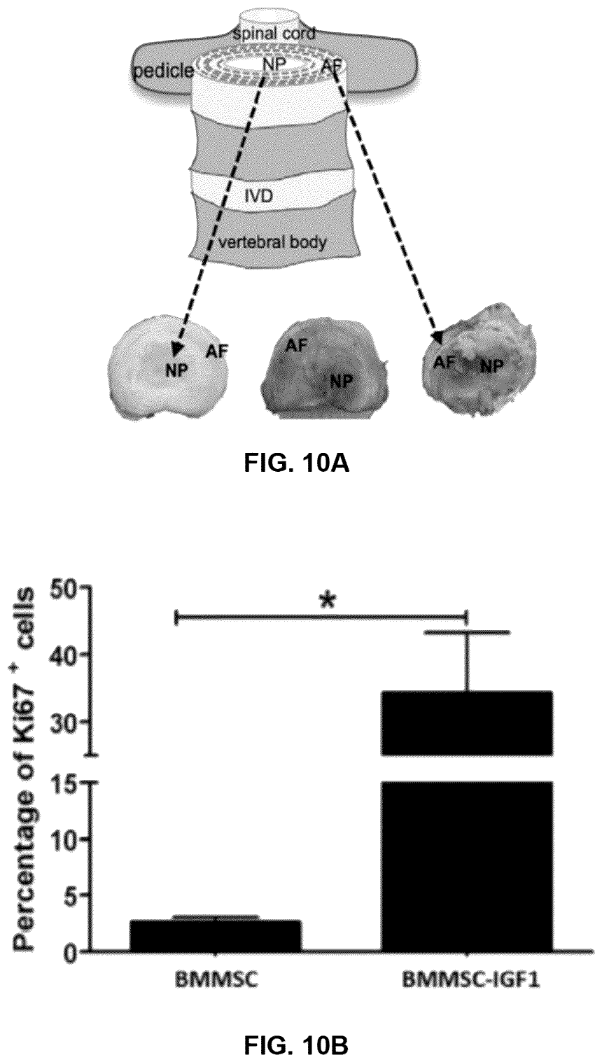

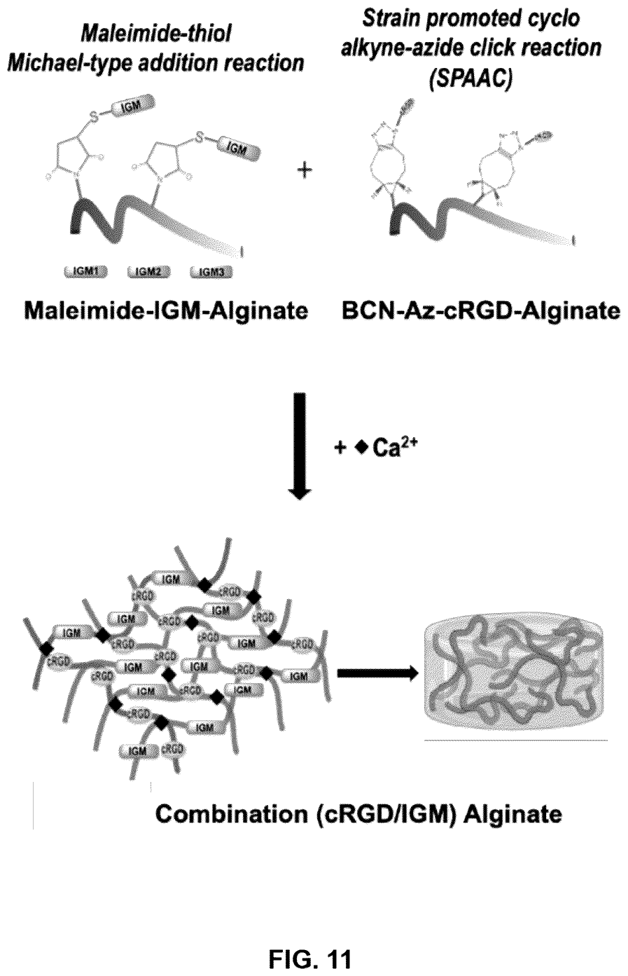

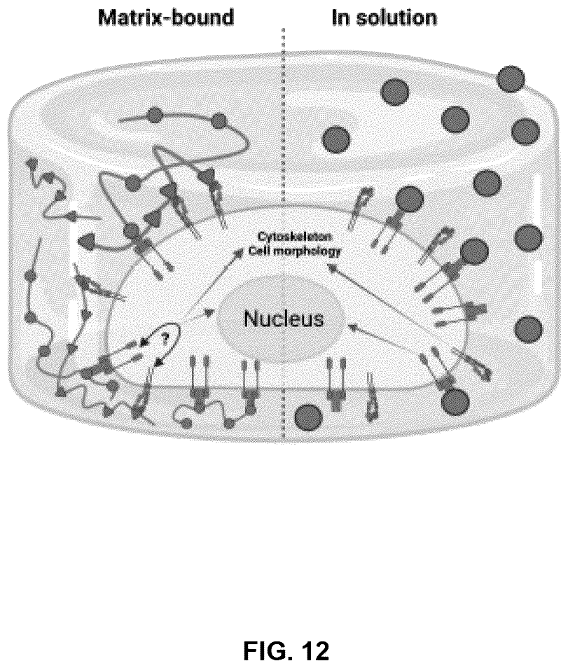

We first screened three candidate peptides that either mimic IGF-1 or the structurally similar growth factor, insulin (FIG. 1A). These peptides were previously identified based on their sequence similarity to IGF-1 or insulin (IGM-1) or through phage-display studies (IGM-2, IGM-3). For screening studies, alginate polymers were grafted with IGF-1 peptide mimetics or cRGD using coupling chemistry described previously (FIG. 1B). MSC were encapsulated into alginate gels via calcium crosslinking. Among the three peptides, IGM-3 best maintained MSC viability in serum-deprived media, with cell viability approaching levels that were observed in cRGD-coupled alginate gels supplemented with a high dose of soluble recombinant human IGF-1 (rhIGF-1 at a dose of 500 ng/mL, FIG. 2A). In contrast, alginate gels coupled with cRGD together with either IGM-1 or IGM-2 elicited only a modest improvement in MSC viability over what was achieved with cRGD-coupled alginate alone. Optimizing co-presentation of cRGD together with IGM-3 further enhanced material performance, to the point that cRGD/IGM-3 functionalized alginates elicited a significant improvement in MSC viability over what was achieved with cRGD and a high dose of soluble IGF-1 (FIG. 2B). Similarly, cRGD/IGM-3-alginate significantly supported MSC viability compared to IGM-3 hydrogel alone, which suggests the presentation of both cell-adhesive and IGM-3 peptide mimetic is necessary to support cell survival under no serum condition (FIG. 6).

IGF-1 triggers pro-reparative behaviors (e.g. proliferation) and blocks apoptosis in MSC and other cell types (e.g. cardiomyocytes and skeletal myocytes) primarily by activating the PI3K/AKT. Since IGM-3 does not share a sequence with IGF-1, we questioned whether it could activate signaling in a similar manner. Thus, we probed the activation of the Akt pathway in MSCs by IGM-3. Surprisingly, short term culture in cRGD and IGM-3 peptide-coupled alginate activated Akt signaling in MSCs to levels similar to culture in cRGD-coupled alginate with the addition of a high dose of soluble IGF-1 (500 ng/mL; FIG. 2C).

To determine if IGM-3 induced Akt signaling activation through the same receptor as soluble IGF-1, cells were cultured atop cRGD-alginate supplemented with soluble IGF-1 or cRGD/IGM-3-alginate gels in the presence of either AZ7550 Mesylate (an active metabolite of AZD9291, IGF-1 receptor inhibitor) or GSK1904529A (IGF-1 receptor and insulin receptor inhibitor). Akt activity was reduced in MSCs on cRGD/IGM-3-alginate treated with IGF-1 receptor inhibitor (AZ7550 mesylate), and decreased further when both receptors were inhibited to a level similar to that observed when incubated with LY294002, an inhibitor of all Akt activation (FIG. 2D, FIG. 2E, FIG. 2F). Importantly, gel-grafted IGM-3 and soluble IGF-1 treated MSC exhibited similar Akt signal modulation via AZ7550 mesylate and GSK1904529A. These observations suggest that IGM-3 activates Akt signaling through similar mechanisms as soluble IGF-1. In contrast to Akt signaling, ERK1/2 signaling was neither promoted nor inhibited significantly by IGM-3, suggesting that cell-extracellular matrix (ECM) adhesion is the predominant means for which this signaling pathway is activated in our gels (FIG. 2G).

Bioactivity of Three IGF-1 Peptide Mimetics Functionalized Alginate in Combination with cRGD Coupling

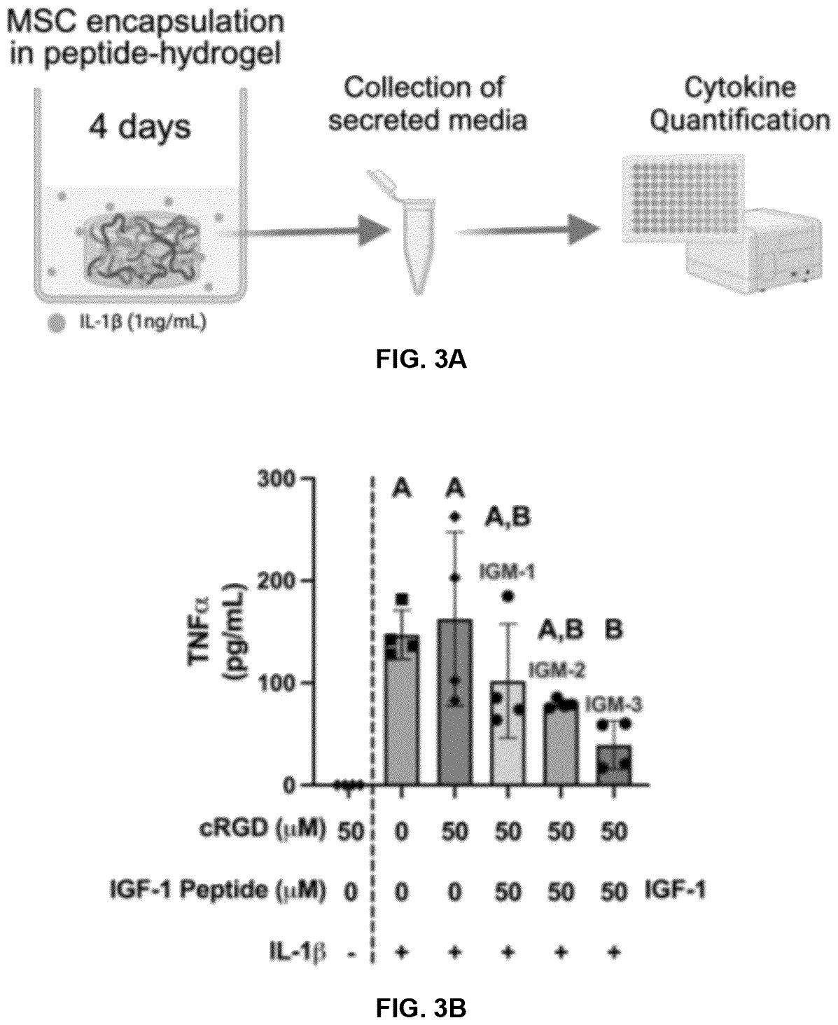

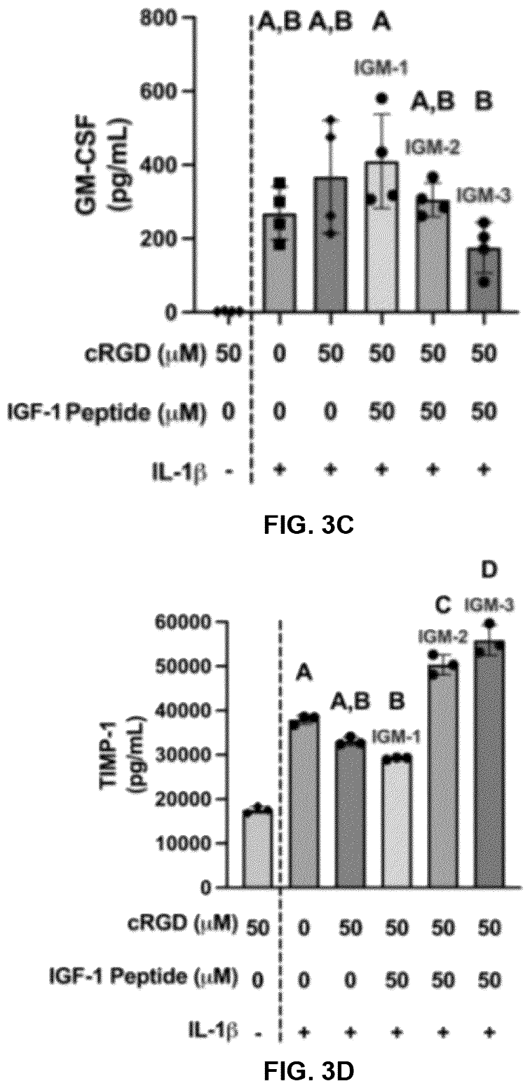

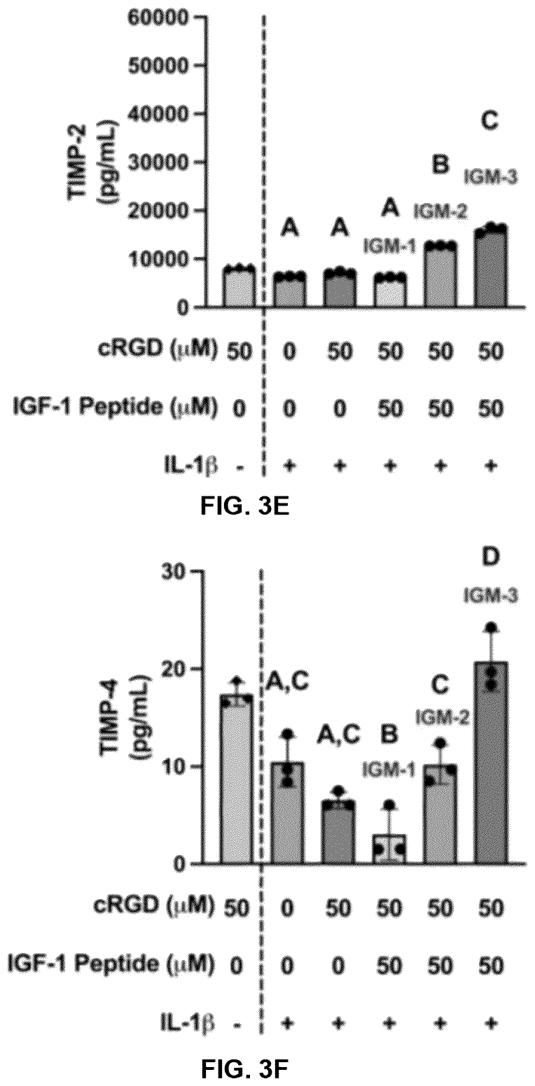

We next assessed the potential of our three candidate peptides to modulate cytokine provoked MSC secretory behavior. IL-1β is present in inflammatory environments into which MSCs are typically transplanted for regenerative applications, and can provoke a pro-inflammatory phenotype in MSC. Consistent with this prior observation, IL-1β induced marked secretion of inflammatory cytokines by MSC during culture (FIG. 3A, FIG. 3B, FIG. 3C, FIG. 3D, FIG. 3E, FIG. 3F). IL-1β increased the secretion of cytokines regardless of hydrogel condition (FIG. 3B, FIG. 3C, FIG. 3D, FIG. 3E, FIG. 3F). However, MSC encapsulated into gels coupled with cRGD and IGM-3 secreted substantially lower levels of TNF-α and granulocyte-macrophage colony-stimulating factor (GM-CSF) compared to MSC cultured in alginate without peptide modifications (“naked” alginate), or alginate modified with cRGD alone (FIG. 3B, FIG. 3C). Furthermore, MSCs cultured within cRGD/IGM-3-alginate secreted the highest levels of Tissue Inhibitor of Metalloproteinases (TIMP 1, 2 and 4) (FIG. 3D, FIG. 3E, FIG. 3F). This suggests the potential that IGM-3 not only blunts IL-1β-induced inflammation in MSC, but that the MSC under these conditions might secrete factors to attenuate ECM degradation in the context of injuries.

Modulation of Cytokine Production in MSC Encapsulated in cRGD/IGM-3 Alginate During Inflammatory Challenge

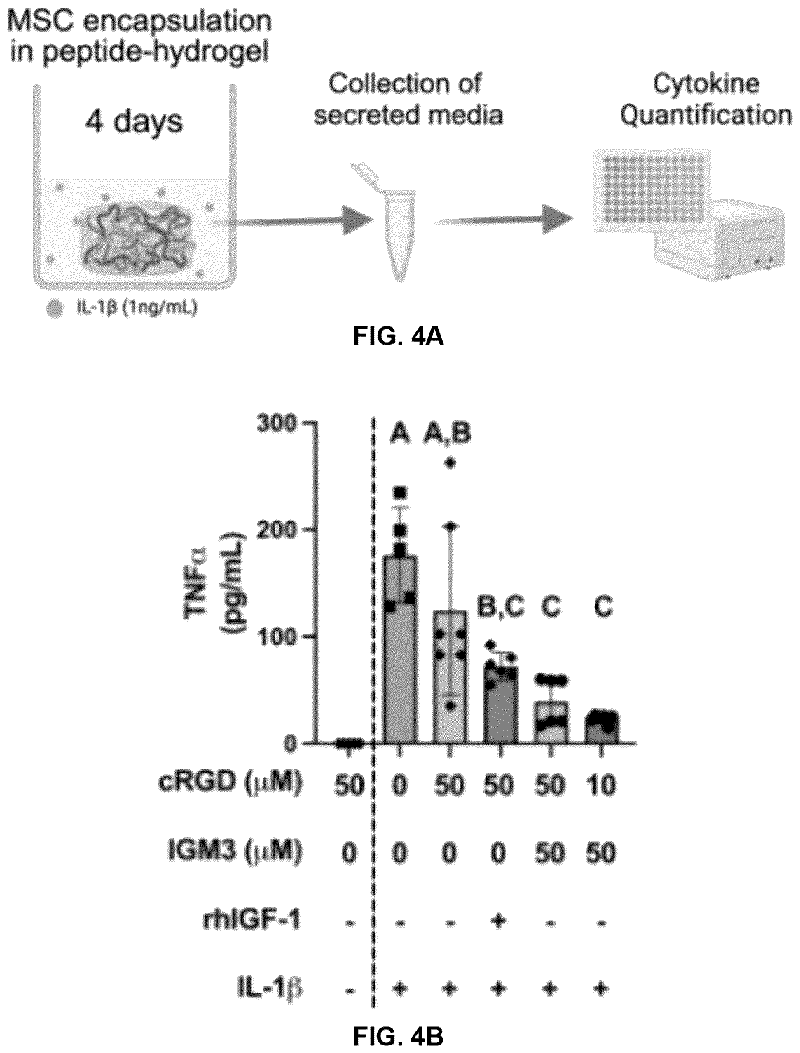

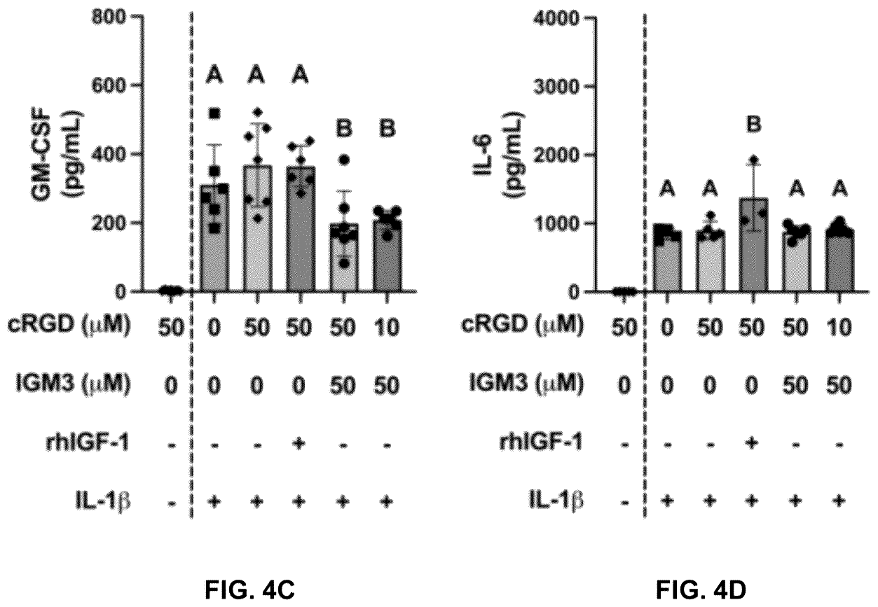

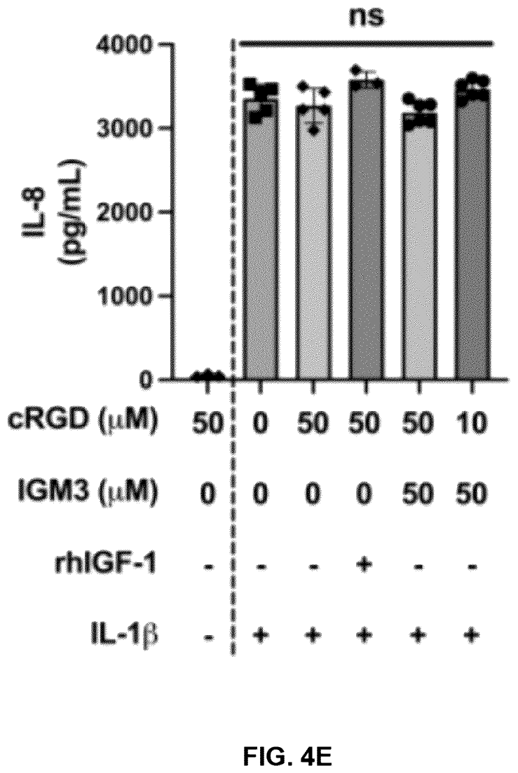

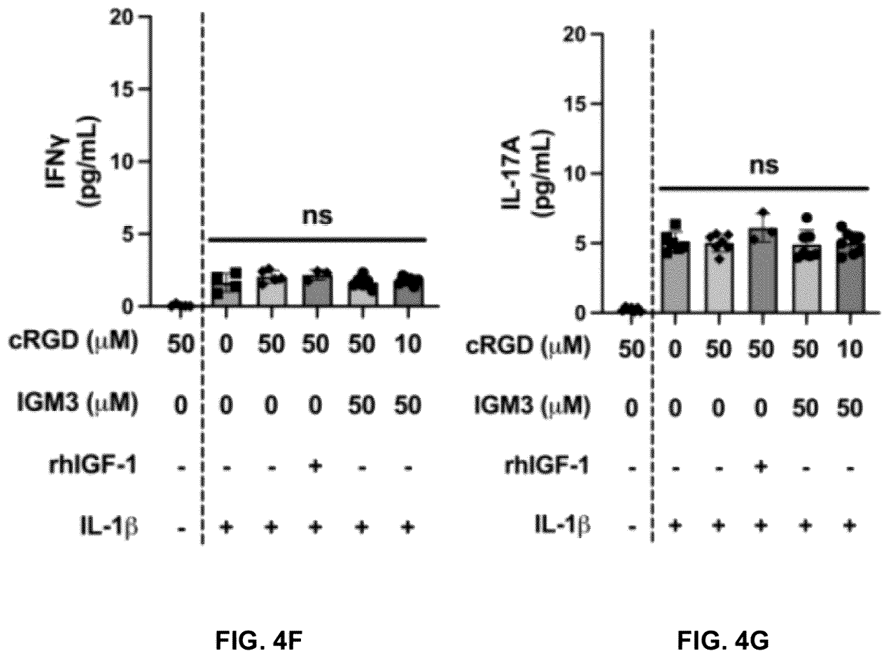

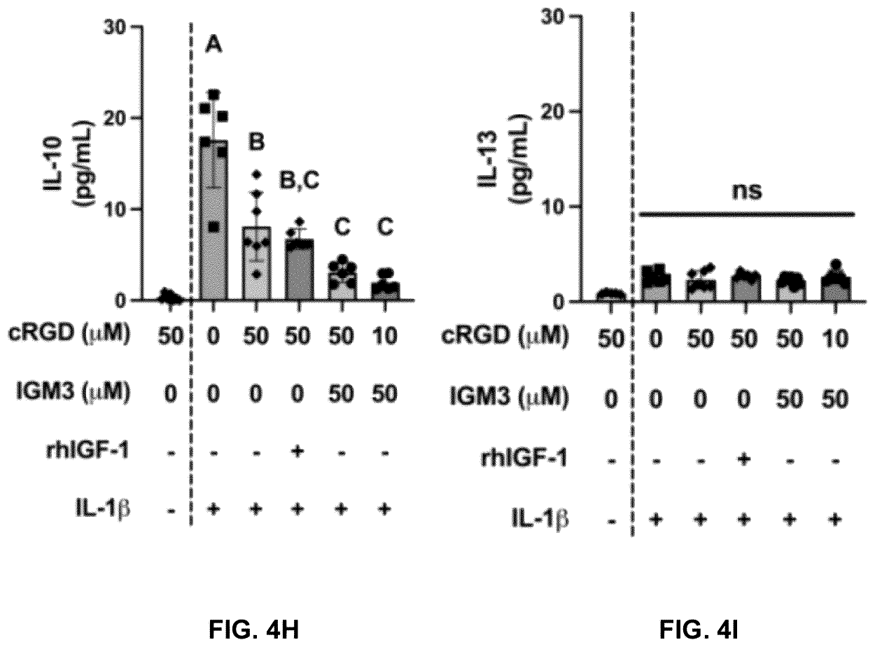

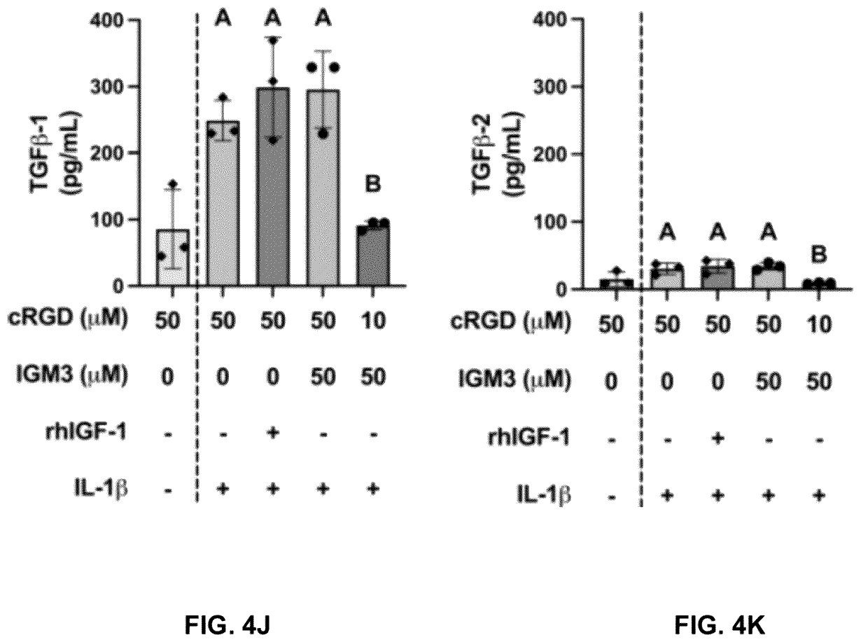

To further explore the immunomodulatory effects of the IGM-3 peptide mimetic under inflammatory conditions, we quantified a broad array of secreted factors linked to pro-inflammatory and anti-inflammatory responses for MSC cultured in the gels presenting cRGD/IGM-3 at the combination yielding optimal survival (FIG. 2B, FIG. 4A). As expected, IL-1β challenge increased MSCs' secretion of pro-inflammatory cytokines, regardless of the alginate hydrogel used for cell encapsulation (FIG. 4B, FIG. 4C, FIG. 4D, FIG. 4E, FIG. 4F, FIG. 4G). Notably, however, MSCs' secretion of TNF-α and GM-CSF were significantly reduced when these cells were encapsulated into cRGD/IGM-3-alginate, with TNF-α reduced to levels similar to that secreted by MSC that were not challenged with IL-1β. (FIG. 4B, FIG. 4C). This reduction in TNF-α and GM-CSF production was similar to the reduction achieved through exposure of MSC to high doses of soluble IGF-1 in the presence of IL-1β (FIG. 4B, FIG. 4C). IL-10 secretion by MSC dramatically increased in unmodified alginate but decreased in both peptide density of cRGD/IGM-3-alginate; the upregulation of IL-10 with IL-1β may be consistent with the cytokine's reported role as regulating both pro and anti-inflammatory responses (FIG. 4H). In contrast to these findings, neither IGM-3 modified gels nor soluble IGF-1 significantly reduced MSCs' secretion of IL-6, IL-8, IFNγ, IL-17A, or IL-1β (FIG. 4D, FIG. 4E, FIG. 4F, FIG. 4G, FIG. 4I).

In addition to inflammation, MSC have also been used to suppress tissue fibrosis, in part by reducing tissue-endogenous TGFβ-1 and TGFβ-2. Consistent with an ability of inflammatory cytokines to induce a pro-fibrotic phenotype in stromal cells like MSC, both TGFβ-1 and TGFβ-2 secretion were markedly upregulated in alginate-encapsulated MSC when treated with IL-1β. However, the encapsulation in the optimal coupling of cRGD/IGM-3-alginate modestly blunted production of these TGF-3 isoforms (FIG. 4J, FIG. 4K); this effect may be more strongly linked to the presence of cell adhesive cRGD as reducing cRGD levels in the alginate gel from 50 μM to 10 μM reduced TGF3 secretion even with IGM-3 levels held constant (FIG. 4J, FIG. 4K).

### IGM-3 Improves MSC's Ability to Diminish Inflammation in Co-Cultured Human Intervertebral Disc (IVD) Cells

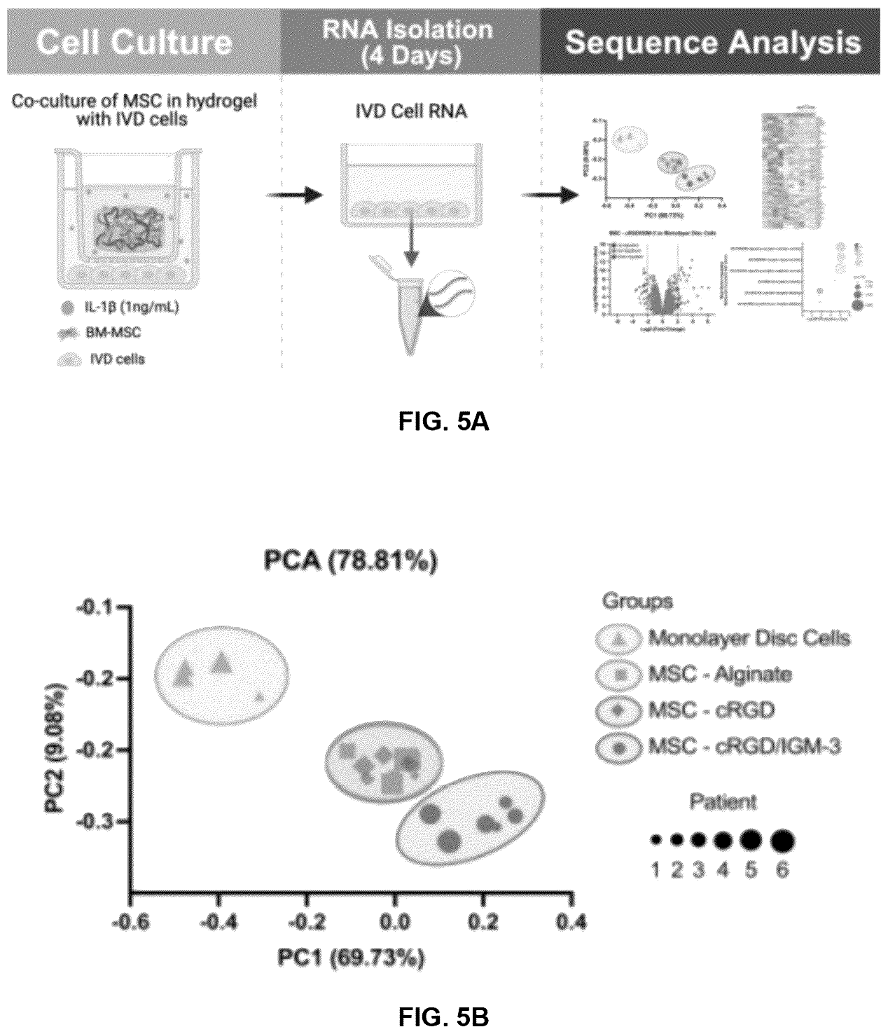

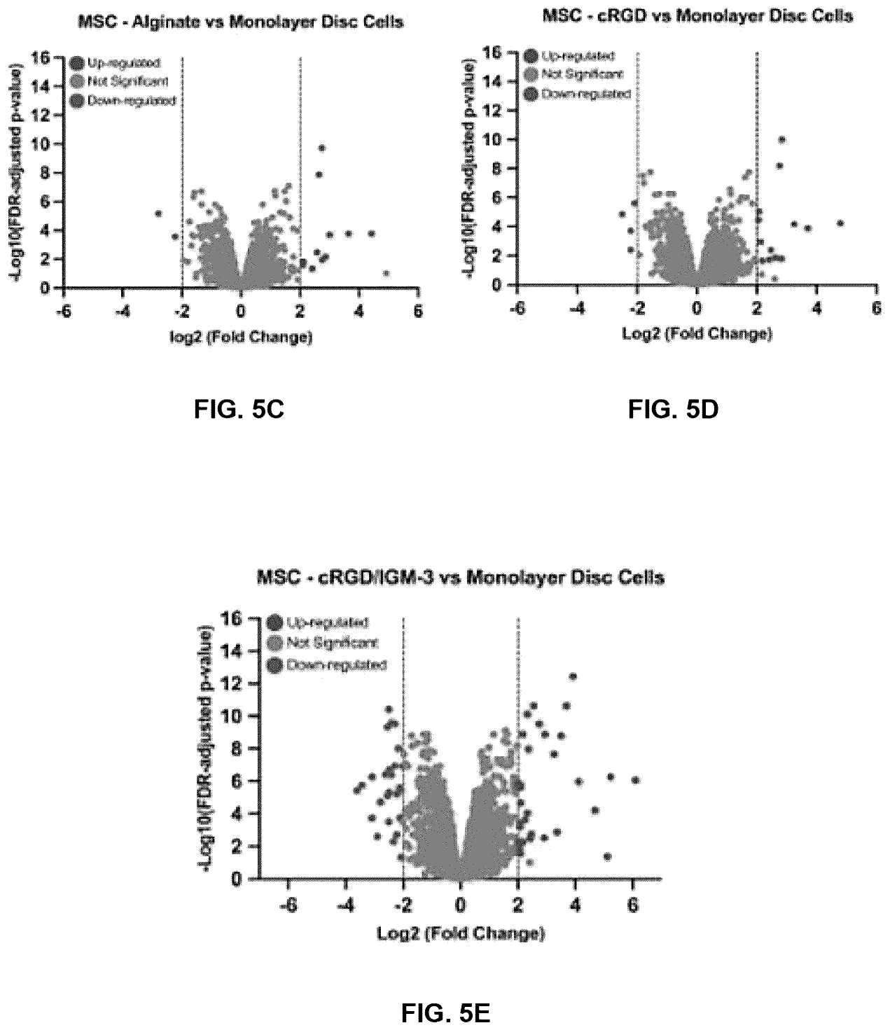

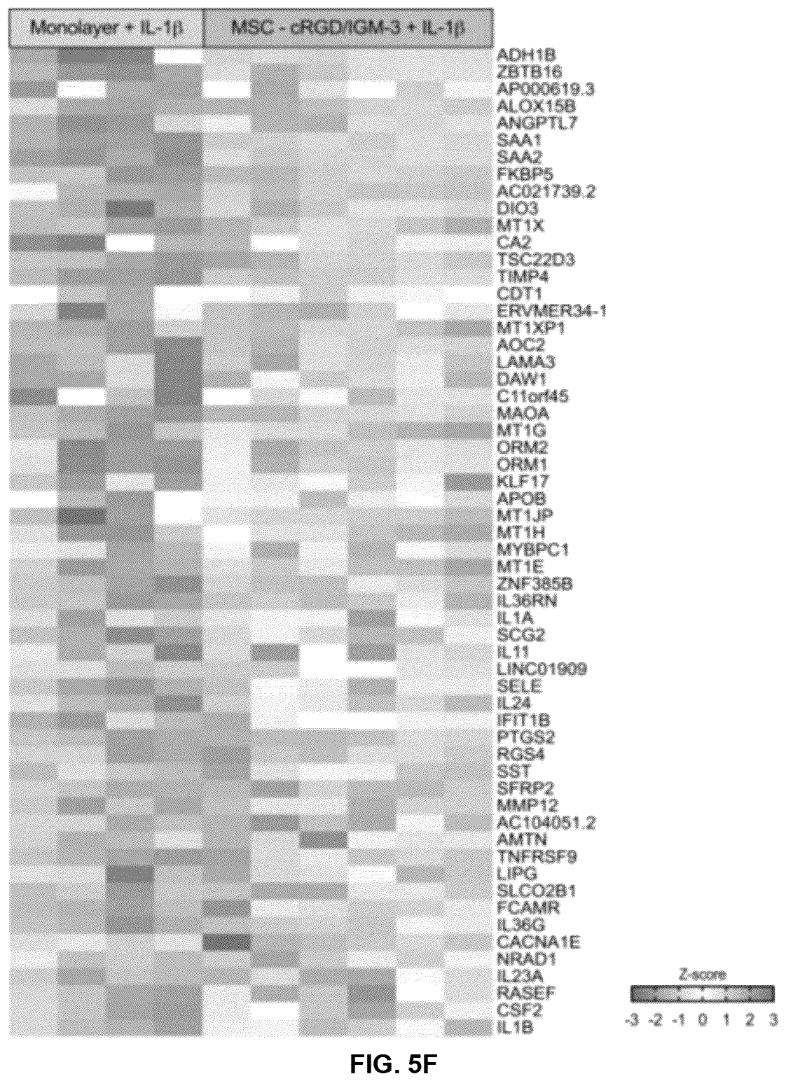

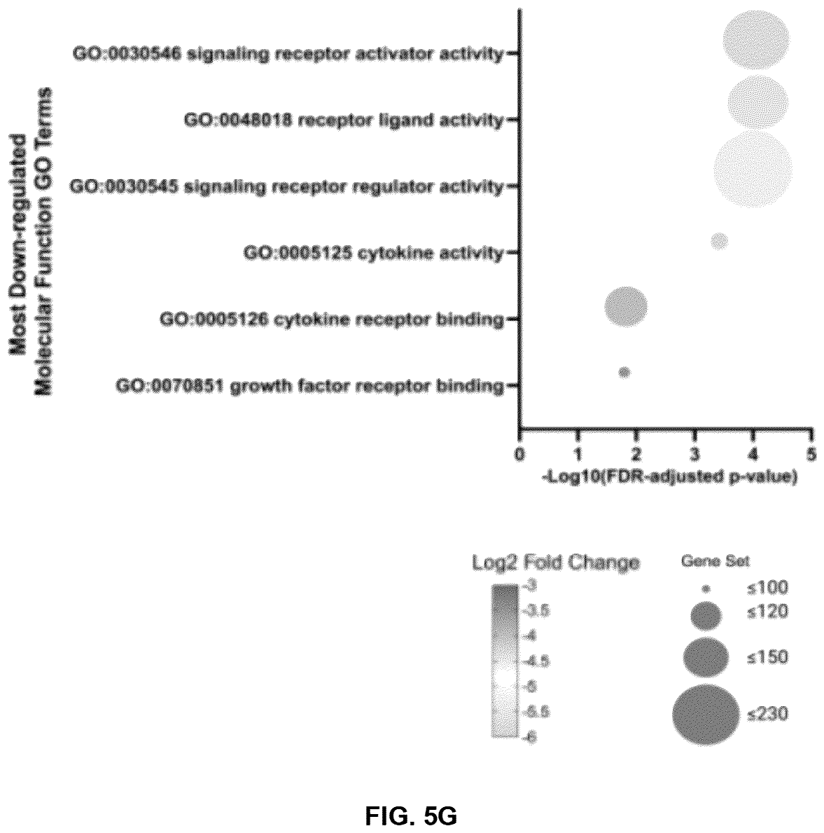

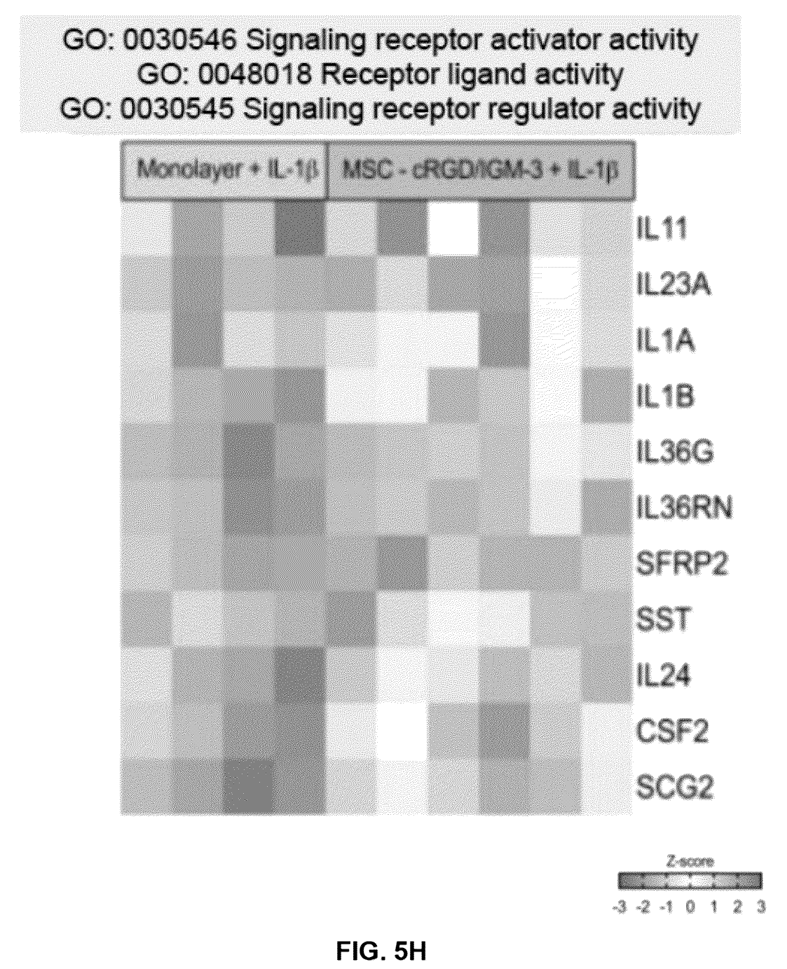

MSCs exert paracrine effects by secreting growth factors and cytokines that enhance recipient tissue regeneration and cellular survival, while dampening immune responses. Thus, we designed a Transwell-based co-culture study where the secretome of gel-encapsulated MSC was exposed to primary cells derived from human degenerative IVD, where both cell populations were challenged with IL-1β. Degenerative IVDs produce a host of pro-inflammatory cytokines, most notably TNF-α and IL-1β and MSC based therapies have long been evaluated in the treatment of this cascade. To obtain a global understanding of how the MSC secretome might ameliorate this pro-degenerative cascade, we profiled these primary human IVD cells using RNA sequencing (FIG. 5A). Analysis showed that while the MSC-secretome can shift global transcription in target IVD cells when MSC were encapsulated into any alginate hydrogel formulation. However, the combined presence of cRGD and IGM-3 in MSC-encapsulated gels had the most dramatic effect on IVD cells (FIG. 5B, FIG. 5C, FIG. 5D, FIG. 5E). This was visualized with Principal Components Analysis (PCA), which showed increased separation between IVD cells exposed to IL-1β without co-culture, versus those exposed to IL-1β in the presence of cRGD/IGM-3-alginate-encapsulated MSC (FIG. 5B). Notably, primary IVD cell populations exposed to IL-1β clustered similarly regardless of whether they were co-cultured with MSC in unmodified alginate gel or MSC in cRGD-alginate. These effects were robust across primary disc cells isolated from 6 patients (3 male, 3 female) who spanned a broad age range (40-70 y.o.). Overlaps in PCA space suggest similar modulatory effects of MSCs in alginate and cRGD alginate on the target disc cells. However, primary disc cells co-cultured with MSC within gels modified with the optimized cRGD/IGM-3 combination clustered distinctly in PCA space (FIG. 5B).

Volcano plot analysis revealed that transcriptional shifts identified in PCA corresponded with a more pronounced differential gene expression in IVD cells co-cultured with MSCs encapsulated in alginate grafted with the optimized combination of cRGD/IGM-3 compared to those in unmodified alginate or cRGD-alginate. This suggests that secreted factors from MSCs in the dual-peptide condition induced significant global phenotypic changes in the IVD cells, particularly in an inflammatory environment (FIG. 5C, FIG. 5D, FIG. 5E). A heatmap using z-score normalized expression displayed a consistent and distinct pattern of downregulated genes related to inflammation (adjusted p-value <0.05, fold change >2 or <−2), highlighting the significant immunomodulatory impact of factors secreted by MSCs within the cRGD/IGM-3 hydrogel (FIG. 5F). This modulation is likely important for enhancing tissue repair mechanisms and controlling inflammatory responses suggesting that MSCs influence not only individual cell behavior but also the broader tissue environment. This finding highlights MSCs' paracrine effects in actively modulating the inflammatory response of cells from a disease state at a transcriptional level. By downregulating pro-inflammatory genes, MSCs exerted a paracrine influence that steers the immune environment towards a reparative state, with potential to enhance tissue repair and attenuate progressive degeneration.

Gene Ontology (GO) analysis provided insights into the molecular function processes that were differentially regulated in IVD cells co-cultured with MSCs in cRGD/IGM-3. Notably, the terms ‘signaling receptor activator activity,’ ‘receptor ligand activity,’ and ‘signaling receptor regulator activity’ were among the top three most significantly regulated by the experimental condition (adjustedp-value <0.05, fold-change >2 or <−2; FIG. 5G). A heatmap using z-score normalized expression for the top three GO terms displayed a consistent pattern of downregulated inflammation-related genes (adjustedp-value <0.05, fold change >2 or <−2), such as IL11, IL23A, IL-1β, and IL1A, and CSF2 (FIG. 5H). This downregulation further highlights the immunomodulatory effect of factors secreted by MSCs within the cRGD/IGM-3 hydrogel. The prominence of these changes suggests that the MSCs, through the cRGD/IGM-3 hydrogel, play a pivotal role in modulating cell signaling pathways critical for cellular communication and response to environmental cues.

### DISCUSSION

Recent works have shown phage-derived peptides can bind specifically to stem cell receptors, enabling their use in functionalizing synthetic scaffolds for stem cell expansion in vitro. These peptides have been incorporated into biomaterials to bind to adhesive or growth factor receptors and shown to promote cell growth and functionality. Our findings demonstrate that the IGM-3 peptide sequence, discovered through phage-display, supports MSC survival and exerts significant immunomodulatory effects in a serum-free environment while two other sequences, IGM-1 and IGM-2 exhibit some but lesser bioactive effects upon MSCs. Importantly, although IGM-3 lacks sequence similarity to natural IGF-1, this peptide mimetic activates downstream signaling similarly to soluble IGF-1 (FIG. 2C), supporting cell survival and cytokine production of MSCs in the presence of IL-1β. Studies with IGF-1 receptor and insulin-receptor specific small molecule inhibitors strongly suggest that the mechanism of action for IGM-3 is similar to that of IGF-1 (FIG. 2D, FIG. 2E, FIG. 2F). This suggests that despite lacking homology to full-length IGF-1, IGM-3's peptide sequence can efficiently engage the IGF-1 receptor or insulin receptor to elicit desired downstream effects to modulate MSC function. The C-domain of IGF-1 (IGM-1) has been previously studied for its role in cell survival and anti-inflammatory signaling in MSCs; however, IGM-3 proved to be more potent for both applications in the present study (FIG. 2A, FIG. 3B, FIG. 3C, FIG. 4B, FIG. 4C). IGM-2 also shown less potency compared to IGM-3 (FIG. 2A), which could be due to its selective binding to the insulin receptor as shown by other studies since insulin receptor is to mediate the metabolic effects of insulin, whereas the IGF-1 receptor is involved in mitogenic signaling. The selective binding of each of the IGM peptide mimetic suggests a different profile with regard to cellular signaling and mitogenic activity.

IGF-1 receptor signaling is well-documented for its critical role in promoting cell survival, proliferation, and immunomodulation through the PI3K/Akt pathway. Our observation also points to a role for IGF-1-induced signaling in immunomodulation in which the addition of soluble IGF-1 or IGM-3 peptide mimetic in combination of cRGD lowered the production of inflammatory cytokines (FIG. 4B). Previous research indicates that IGF-1 can suppress the expression of toll-like receptor 4 in skeletal muscle cells, reducing inflammation via the PI3K/Akt pathway and consequently lowering TNF-α and IL-6 levels. In addition to its effects through the PI3K/Akt pathway, IGF-1 has been shown to modulate MSCs by activating the JAK/STAT pathway, thereby enhancing their survival and anti-inflammatory capabilities. For example, Takahashi et al. and colleagues elucidated the activation of JAK/STAT signaling pathways by IGF-1 in rat cardiomyocytes, in which there is a complex interplay of kinase activities and phosphorylation events essential for cellular responses to growth signals. Based on these prior results, it is plausible that culturing MSC within the cRGD/IGM-3-hydrogels, which markedly reduced both pro-inflammatory cytokines (e.g. TNF-α) but also variably affected anti-inflammatory cytokines (e.g. IL-10; FIG. 4B, FIG. 4H), activated JAK/STAT to antagonize inflammatory signaling pathways.

The therapeutic efficacy of MSCs in exerting trophic effects on endogenous cells upon transplantation within inflammatory environments is predominantly attributed to paracrine signaling rather than direct cell integration. Our findings revealed that regardless of the micro-environment that MSC were encapsulated into (e.g. unmodified alginate vs. cRGD vs. cRGD/IGM-3), the secretome of these cells strongly affected global RNA expression of primary cells derived from degenerative human IVD that were provoked with IL-1 (FIG. 5C, FIG. 5D, FIG. 5E). However, the most dramatic RNA expression shifts were observed in disc cells co-cultured with MSC encapsulated into cRGD/IGM-3-alginate (FIG. 5C, FIG. 5D, FIG. 5E). Notably, we observed a downregulation of genes such as TNFRSF9, CSF2, and IL-1β, which are associated with anti-inflammatory responses, suggesting a shift towards a less inflammatory state in IVD cells co-cultured with MSCs within the cRGD/IGM-3-alginate (FIG. 5F). These paracrine effects of MSCs in modulating a targeted endogenous cell population in vitro are consistent with prior reports. For example, Levorson et al. demonstrated that chondrocyte proliferation and expression of ECM proteins like aggrecan that are associated with healthy cartilage were enhanced when the chondrocytes were co-cultured with MSC. Other studies have demonstrated that MSCs co-cultured with degenerated nucleus pulposus cells of the IVD upregulated TGFβ-1, IGF-1, and aggrecan gene expression in these cells, while MSCs had no effect on the phenotype of non-degenerate nucleus pulposus cells. This suggests that secreted factors by MSCs could stimulate the endogenous IVD cell population to regain a nondegenerative phenotype.

Taken together, our study illustrates the significant potential of IGF-1 peptide mimetic and cell adhesive ligand in enhancing the survival and immunomodulatory properties of MSCs. MSC within the cRGD/IGM-3-alginate secreted factors that downregulated inflammatory gene expression in primary human degenerative IVD cells, which are typically highly inflammatory during degeneration. These findings illustrate the design of a bioactive biomaterial to retain and enhance stem cell functionality using IGF-1 peptide mimetics and circumvent a potential need to co-administer the cells together with high doses of soluble growth factors. Future work could focus on exploring the synergism between JAK/STAT, PI3K/AKT, and cell adhesive signaling pathways and cellular mechanisms that influence MSC immunomodulation will offer deeper mechanistic insights into the potential for co-delivery of MSCs and growth factor peptide mimetics in the development of MSC therapies.

### Materials and Methods

### Peptide-Modified Hydrogel

High-molecular-weight, high G-block-containing alginate (Manugel, Dupont, Wilmington, De, USA) was dissolved at 1% (w/v) solution in Dulbecco's PBS (dPBS). Alginate polymer solution was purified by dialyzing against deionized water. For peptide conjugation to alginate, Alginate was modified with N-terminal cysteine presenting IGM peptides using maleimide-thiol click chemistry. Briefly, to graft each of the cysteine-terminated IGM peptides (FIG. 1A, GenScript, Piscataway, NJ), the maleimide-modified alginate was dissolved at 1% w/v in ultrapure water for 2 h. Either IGM-1 (CGGGYGSSSRRAPQT SEQ ID NO:1), IGM-2 (CGGGSLDESFYDWFERQLGKK SEQ ID NO:2), or IGM-3 (CGGGDYKDLCQSWGVRIGWLAGLCPKK SEQ ID NO:3) was dissolved in ultrapure water and added dropwise to the maleimide-modified alginate solution at an equivalent to twice the molar ratio of BMPH conjugated to the alginate polymers [e.g., when the measured molar output for maleimide was 5 maleimide/polymer, a molar input of 10 IGM-polymer was used]. Alginate was modified with cRGD peptides coupled through a heterobifunctional spacer using Strain Promoted Azide Alkyne Cycloaddition (SPAAC) click chemistry. Briefly, BCN-alginates were dissolved at 1% w/v in Dulbecco's phosphate-buffered saline (dPBS) for 2 h. Azide cRGD (cyclo[Arg-Gly-Asp-D-Phe-Lys-(azide)]; Peptides International; Louisville, KE, USA) was then added dropwise to BCN-alginate solution at an equivalent to twice the molar ratio of BCN amine conjugated to the alginate polymer and allowed to react at room temperature with constant stirring for 24 h. Following dialysis (3400 Da cutoff) for 72 h, polymers were sterile filtered (0.42 μm) and freeze-dried polymers were maintained under aseptic conditions and stored at −80° C. for further use. Polymers prepared for subsequent studies were unmodified alginate, 10 μM cRGD-alginate, 50 μM cRGD-alginate, 50 μM cRGD/50 μM IGM-1, 50 μM cRGD/50 μM IGM-2, 50 μM cRGD/50 μM IGM-3, or 10 μM cRGD/50 μM IGM-3. Polymer solution was ionically crosslinked using divalent calcium ions and used for subsequent studies.

| IGF-1 peptide | Cysteine terminated linker | Method of | Putative |
|---|---|---|---|
| mimetic | and peptide sequence | Identification | Receptor |
| IGM3 | CGGGDYKDLCQSWGVRIGWLAGLC | Phage- | IGF-1R |
| | PKK (SEQ ID NO: 3) | display | and/or IR |
| IGM3 | CGGGLCQSWGVRIGWLAGLCPKK | Phage- | IGF-1R |
| (No Flag-tag) | (SEQ ID NO: 4) | display | and/or IR |

### Human Bone-Marrow Derived Mesenchymal Stem Cell (MSC) Culture

Human bone marrow-derived MSC (RoosterBio™, Frederick, MD) were acquired and expanded according to RoosterBio™ expansion protocol. Briefly, MSCs were plated in tissue culture flasks in Roosterbasal™ medium supplemented with RoosterBooster™ until 80% confluence at 37° C. and 5% C02. MSCs were detached using Gibco TrypLE™ Express Enzyme (1×) (ThermoFisher Scientific, Waltham, MA) and passaged. Cells from passages 2 to 5 were used for experiments to ensure their multipotent characteristics.

### Cell Survival Assessment

Sterile, freeze-dried polymers were dissolved in 100 mM HEPES buffer (pH 7.2) at 2% w/v concentration and mixed overnight at 4° C. on a rotator. MSCs were prepared at a density of 2.5×10\hat{ }6 cells/mL and mixed with an equal volume of the alginate solution to achieve a final concentration of 1% w/v alginate in the hydrogels. These hydrogels were then crosslinked using a calcium sulfate slurry to a final concentration of 100 mM calcium. The mixtures were quickly poured between two glass plates spaced 1 mm apart and left at room temperature for one hour to complete the crosslinking process. The formed hydrogels were punched into 5 mm discs using a biopsy puncher and placed in a 96-well plate containing RoosterBasa™ medium supplemented with RoosterBooster™ for 24 hours, then switched to RoosterBasal™ medium alone for 7 days at 37° C. and 5% C02, with a 50% media change every 48 hours. Human recombinant insulin-like growth factor-1 (rhIGF-1, 500 ng/mL, Peprotech, Cranbury, NJ) was added to the unmodified alginate and cRGD-alginate hydrogel as positive controls.

Cell viability was assessed using the Resazurin-based Alamar Blue assay (Thermo Fisher Scientific). After culturing in RoosterBasal™ for 4 days, all hydrogel conditions encapsulated with MSCs were gently rinsed with phosphate-buffered saline (PBS). Fresh medium containing 10% Alamar Blue solution was then added to each well, and the plates were incubated for an additional 4 hours at 37° C. in a humidified atmosphere with 5% CO₂. The fluorescence intensity of the medium was measured spectrofluorometrically at 560 nm/590 nm excitation/emission using an EnSight Multimode Plate Reader (PerkinElmer, Waltham, MA). The Resazurin readouts on day 7 were normalized to the readings obtained at 24 hours from the same gel, with each gel treated as a biological replicate.

### Signaling Pathway Activation Studies

Both non-phosphorylated and phosphorylated forms of the proteins AKT1/2/3, p-AKT 1/2/3 (pS473), total ERK 1/2, and p-ERK 1/2 (Thr202/Tyr204) were quantified using AlphaLISA SureFire Ultra Assay Kits (high volume, Revvity, Waltham, MA). For this purpose, sterile polymers, specifically 10 μM cRGD-alginate, and 10 μM cRGD/50 μM IGM-3-alginate, were dissolved at 1% w/v in sterile 100 mM HEPES buffer (pH 7.2) and stirred continuously overnight at 4° C. Subsequently the next day, these solutions were rapidly mixed with sterile calcium sulfate slurry to achieve a final concentration of 100 mM calcium in the gel. The resulting mixtures were then poured between two glass plates separated by 1 mm spacers and allowed to crosslink at room temperature for one hour. Following crosslinking, the hydrogels were punched into 5 mm discs using a biopsy punch and transferred to wells of a 96-well plate containing RoosterBasal™ medium. These gels were incubated at 37° C. and 5% C02, with the incubation medium refreshed six times at regular intervals over three days to remove excess calcium before cell seeding. Prior to the signaling assay, the medium in the culture flasks containing MSCs was replaced with RoosterBasal™ without RoosterBooster™ supplementation for 24 hours. MSCs were then detached using Gibco TrypLE™ Express Enzyme (1×) (ThermoFisher Scientific), resuspended in RoosterBasa™, and seeded onto the gel at a density of 50,000 cells per well. After cell seeding, treatments were administered to designated wells, including AZ7550 Mesylate (20 PM, MedChem Express), GSK1904529A (3 μM, MedChem Express), LY294002 (10 μM, MedChem Express), U0126 (10 μM, MedChem Express), rhIGF-1 (500 ng/mL, Peprotech), or no treatment. Cells were allowed to attach for 2 hours at 37° C. and 5% C02, after which each cell-seeded hydrogel was considered an individual sample for subsequent analysis. The AlphaLISA assay was then performed according to the manufacturer's protocol. Briefly, the medium was removed, and lysis buffer was added to each well. AlphaLISA reaction buffers (donor and acceptor mixes) were subsequently added to the lysate before the wells were read using an EnSpire Multimode Plate Reader (PerkinElmer) with the AlphaScreen protocol at an excitation of 680 nm and emission of 615 nm. Maximal protein phosphorylation was quantified by the ratio of phosphorylated protein to total protein in MSCs for p-AKT1/2/3 (pSer473) and p-ERK1/2 (pThr202/pTyr204).

### Multiplexed Secreted Protein Analyses

MSCs were encapsulated in each alginate gel as described Cell Survival Assessment Section. Following initial encapsulation, the hydrogel-containing cells were maintained in RoosterBasa™ medium and treated with 1 ng/mL of IL-1 (Sigma) for 4 days at 37° C. and 5% CO₂. After the treatment period, cell culture supernatants were collected, transferred to tubes, and stored at −80° C. until analysis. Protein levels of pro-inflammatory cytokines and chemokines in the supernatants were quantified using Luminex xMAP technology. The multiplex analysis was performed using a Luminex™ 200 system (Luminex, Austin, TX) by Eve Technologies Corp. (Calgary, Alberta, Canada) with human multiplex kits from MilliporeSigma (Burlington, Massachusetts, USA).

### Primary Human Intervertebral Disc (IVD) Cell Culture

Intervertebral disc cells (n≥3, male and female, ages 40 to 70) were isolated from the nucleus pulposus region of to-be-discarded surgical waste tissues; patients were de-identified and only sex, race and age of the patient were recorded (non-human subjects research exempt from pathology review; approved by Washington University IRB). For these studies, disc tissue was isolated from 6 patients (3 male, 3 female) aged 40-70.

Disc cells were isolated as described. Briefly, tissue fractions were removed from surgical samples and digested for 4 h at 37° C. and 5% CO₂ in medium containing 0.4% type II collagenase. (Worthington Biochemical, Lakewood, NK) and 2% pronase (Roche, Basel, Switzerland). Cells were passed through a 70 μm cell strainer (Thermo Fisher Scientific, Waltham, MA). The isolated cells (PO) were plated in tissue culture flasks in Ham's F12 medium (Thermo Fisher Scientific) supplemented with 10% fetal bovine serum (FBS) and 1% penicillin-streptomycin (P/S) until confluence. For co-culture experiments, primary human disc cells were seeded in the bottom wells of 24-well Corning™ Transwell™ plates (Corning, NY, USA) with 0.4 μm permeable polyester membrane inserts. These cells were cultured at a density of 30,000 cells per well in F12 medium containing 10% FBS and 1% pencillin-streptomycin under hypoxic condition (37° C. and 5% CO₂ and 2% 02) for 24 h.

### In Vitro Transwell Co-Culture of Monolayer IVD Cells and MSCs Encapsulated in Hydrogel

At 24 hours, the encapsulation of bone marrow-derived MSCs was performed as previously described. MSCs were encapsulated within alginate, 10 μM-cRGD, or 10 μM-cRGD/50 μM-IGM-3 alginate hydrogels. The Transwell inserts were pre-moistened with RoosterBasal™ medium, and each hydrogel was then placed within a Transwell insert. These inserts were subsequently positioned in wells containing primary human disc cells, which were cultured in RoosterBasal™ medium treated with 1 ng/mL IL-1β3 under hypoxic (2% 02) conditions to challenge both the MSC and IVD cells. Monolayer IVD cells with no co-culture treated with 1 ng/mL IL-1β served as control. All wells were incubated for 4 days, with 50% media changes conducted at 48-hour intervals.

### Cell Lysate Collection and RNA Processing

Following the 4-day incubation period, hydrogels containing MSCs were removed from the Transwell inserts. IVD cells that were attached to the well were washed with 1% dPBS and lysed using RLT buffer (Qiagen, Hilden, Germany) and 1% mercaptoethanol (Sigma). mRNA isolation was performed using the RNeasy Kit with DNase I digestion (Qiagen Iberia, Madrid, Spain), following the manufacturer's protocol. Briefly, NP cell samples were homogenized using a QlAshredder™ column, processed through an RNeasy spin column for RNA binding, followed by washing, and DNA digestion with DNase I. The purified RNA was eluted after two washes with washing buffer. The quality and quantity of the isolated mRNA were assessed by measuring the absorbance ratio at 260/280 nm using a NanoDrop One Spectrophotometer (Thermo Fisher Scientific).

cDNA Library Generation and Bulk RNA-Sequencing (Bulk RNA-Seq) with Data Standardization

The input samples were submitted to the Washington University Genome Technology Access Center to obtain and sequence the cDNA libaraies (Illumina NovaSeq X Plus) according to manufacturer's protocol. Briefly, RNA-seq reads were aligned to the Ensembl release 101 primary assembly with STAR version 2.7.9a. Gene counts were derived from the number of uniquely aligned unambiguous reads by Subread: feature Count version 2.0.3. Isoform expression of known Ensembl transcripts were quantified with Salmon version 1.5.2. Sequencing performance was assessed for the total number of aligned reads, total number of uniquely aligned reads, and features detected. The ribosomal fraction, known junction saturation, and read distribution over known gene models were quantified with RSeQC version 4.0.

All gene counts were imported into the R/Bioconductor package EdgeR and TMM normalization size factors were calculated to adjust samples for differences in library size. Ribosomal genes and genes not expressed in the smallest group size minus one samples greater than one count-per-million were excluded from further analysis. The TMM size factors and the matrix of counts were then imported into the R/Bioconductor package Limma. Weighted likelihoods based on the observed mean-variance relationship of every gene and sample were then calculated for all samples and the count matrix was transformed to moderated log 2 counts-per-million with Limma's voom With Quality Weights. The performance of all genes was assessed with plots of the residual standard deviation of every gene to their average log-count with a robustly fitted trend line of the residuals. PCA was conducted using the prcomp function in R (RStudio Team 2020) to reduce dimensionality of the data. Differential expression analysis was then performed to analyze for differences between conditions in NP cells or MSCs, respectively. The results were filtered for only those genes with Benjamini-Hochberg false-discovery rate adjusted p-values less than or equal to 0.05 and an absolute log 2 fold change equal to 2 or greater. Gene Ontology analysis was conducted for differentially expressed genes using the clusterProfiler package (v3.14.3) in R. Results were filtered for terms with an adjusted p-value less than or equal to 0.05 and log 2 fold-change less than −2 or greater than 2.

### Statistical Analysis

Statistical analyses were performed using GraphPad Prism 9 (Graph Pad Software, Inc., San Diego, CA, USA). Significance was determined by one-way ANOVA with Tukey's post hoc test (P<0.05). Two-tailed Student's t-tests were used to estimate statistically significant differences in Akt phosphorylation between 10 μM cRGD-alginate supplemented with rhIGF-1 and 10 μM cRGD/50 μM IGM-3-alginate combination. Data represent the mean±SD. Different letter denotes statistical significance.

What is claimed is:

1. A hydrogel composition for culturing mesenchymal stem cells (MSCs), the composition comprising an alginate polymer, a cyclic RGD (cRGD) peptide conjugated to the alginate polymer, and an Insulin-like growth factor (IGF)-1 peptide mimetic conjugated to the alginate polymer.

2. The hydrogel composition of claim 1, wherein the IGF-1 peptide mimetic is IGM3, an amino acid sequence comprising LCQSWGVRIGWLAGLCPKK (SEQ ID NO:3) and variations thereof.

3. The hydrogel composition of claim 1, wherein the hydrogel is formed by ionically crosslinking the alginate polymer using a calcium sulfate solution.

4. The hydrogel composition of claim 1, wherein the cRGD peptide has a concentration in the hydrogel ranging from about 10 μM to about 50 μM.

5. The hydrogel composition of claim 1, wherein the IGF-1 peptide mimetic has a concentration in the hydrogel ranging from about 10 μM to about 50 βM.

6. The hydrogel composition of claim 1, wherein the hydrogel is configured to encapsulate a plurality of MSCs.

7. The hydrogel composition of claim 6, wherein the MSCs are human MSCs.

8. A method of treating a disease or condition comprising administering, to a subject in need, a therapeutically effective amount of a hydrogel composition comprising a hydrogel comprising an alginate polymer, a cyclic RGD (cRGD) peptide conjugated to the alginate polymer, an Insulin-like growth factor (IGF)-1 peptide mimetic conjugated to the alginate polymer, and a plurality of mesenchymal stem cells (MSCs) embedded in the hydrogel.

9. The method of claim 8, wherein the hydrogel is a functionalized alginate hydrogel.

10. The method of claim 9, wherein the functionalized alginate hydrogel has a concentration of about 1% w/v alginate.

11. The method of claim 8, wherein the IGF-1 mimetic is IGM3 comprising the amino acid sequence LCQSWGVRIGWLAGLCPKK (SEQ ID NO:3) and variations thereof.

12. The method of claim 8, wherein the disease or condition is intervertebral disc degeneration.

13. A method of enhancing stem cell function comprising encapsulating mesenchymal stem cells (MSCs) within a hydrogel composition comprising an alginate polymer, a cyclic RGD (cRGD) peptide conjugated to the alginate polymer, and an Insulin-like growth factor (IGF)-1 peptide mimetic conjugated to the alginate polymer.

14. The method of claim 13, wherein the IGF-1 mimetic is IGM3 comprising the amino acid sequence LCQSWGVRIGWLAGLCPKK (SEQ ID NO:3) and variations thereof.

15. The method of claim 13, wherein the hydrogel composition reduces the production of pro-inflammatory cytokines by the embedded MSCs.

16. The method of claim 15, wherein the pro-inflammatory cytokines are selected from tumor necrosis factor (TNF)-α, granulocyte-macrophage colony-stimulating factor (GM-CSF), and any combination thereof.

17. The method of claim 13, wherein the hydrogel composition increases the survival of the embedded MSCs.

18. The method of claim 13, wherein a concentration of the cRGD peptide in the hydrogel ranges from about 10 μM to about 50 μM.

19. The method of claim 13, wherein a concentration of the IGF-1 peptide mimetic in the hydrogel ranges from about 10 μM to about 50 μM.


SEQUENCE LISTING (4 sequences)

SEQ ID NO: 1 - protein, 15 aa, unidentified
CGGGYGSSSRRAPQT

SEQ ID NO: 2 - protein, 21 aa, unidentified
CGGGSLDESFYDWFERQLGKK

SEQ ID NO: 3 - protein, 27 aa, unidentified
CGGGDYKDLCQSWGVRIGWLAGLCPKK

SEQ ID NO: 4 - protein, 23 aa, unidentified
CGGGLCQSWGVRIGWLAGLCPKK